SEQUENCE LISTING (22 sequences)

SEQ ID NO: 1 - DNA, 22 bp, Artificial Sequence
  Synthetic primer
ctgctgtaacgatgaagccctg

SEQ ID NO: 2 - DNA, 22 bp, Artificial Sequence
  Synthetic primer
gctgtaggaagctcatctctcc

SEQ ID NO: 3 - DNA, 22 bp, Artificial Sequence
  Synthetic primer
actgggcaacaccaagtccgaa

SEQ ID NO: 4 - DNA, 23 bp, Artificial Sequence
  Synthetic primer
cacattggctgtgttcttccagg

SEQ ID NO: 5 - DNA, 22 bp, Artificial Sequence
  Synthetic primer
cctgaatcctgggaaatgtgcc

SEQ ID NO: 6 - DNA, 22 bp, Artificial Sequence
  Synthetic primer
cgattcgcacacggtcttctgt

SEQ ID NO: 7 - DNA, 22 bp, Artificial Sequence
  Synthetic primer
aaccgagcctactcacagtacg

SEQ ID NO: 8 - DNA, 22 bp, Artificial Sequence
  Synthetic primer
gcatccttcgtgctgaaatcgg

SEQ ID NO: 9 - DNA, 23 bp, Artificial Sequence
  Synthetic primer
gaactgaggacgcttccacattc

SEQ ID NO: 10 - DNA, 22 bp, Artificial Sequence
  Synthetic primer
tcagaaacgccaacagcacggt

SEQ ID NO: 11 - DNA, 22 bp, Artificial Sequence
  Synthetic primer
tggaccttccaggatgaggaca

SEQ ID NO: 12 - DNA, 22 bp, Artificial Sequence
  Synthetic primer
gttcatctcggagcctgtagtg

SEQ ID NO: 13 - DNA, 22 bp, Artificial Sequence
  Synthetic primer
gtacctctgcaaggtggaactc

SEQ ID NO: 14 - DNA, 23 bp, Artificial Sequence
  Synthetic primer
ccaaaggaggaagtcagaatccg

SEQ ID NO: 15 - DNA, 20 bp, Artificial Sequence
  Synthetic primer
accctggtcattcacttggg

SEQ ID NO: 16 - DNA, 21 bp, Artificial Sequence
  Synthetic primer
catttgctccctctgacactg

SEQ ID NO: 17 - DNA, 22 bp, Artificial Sequence
  Synthetic primer
cttctgctccttgcctgtgtct

SEQ ID NO: 18 - DNA, 22 bp, Artificial Sequence
  Synthetic primer
ggttagtgtcccttcctgccaa

SEQ ID NO: 19 - DNA, 22 bp, Artificial Sequence
  Synthetic primer
ctgcgattctcctggctgtgaa

SEQ ID NO: 20 - DNA, 23 bp, Artificial Sequence
  Synthetic primer
caacaaccataggcgatttctgg

SEQ ID NO: 21 - DNA, 22 bp, Artificial Sequence
  Synthetic primer
aggagtctaacaacggcagcct

SEQ ID NO: 22 - DNA, 22 bp, Artificial Sequence
  Synthetic primer
gtggtacacctcagtctcgaag

(19) United States
(12) Patent Application Publication
(10) Pub. No.: US 2023/0332245 A1
(43) Pub. Date: Oct. 19, 2023

(54) Complement as Prognostic and Predictive Biomarker and Potential Therapeutic Target in Renal Cell Carcinoma

(71) Applicant: Texas Tech University System, Lubbock, TX (US)

(72) Inventors: Maciej J. Markiewski, Abilene, TX (US); Britney Reese, Lubbock, TX (US); Elizabeth Daugherity, Abilene, TX (US)

(21) Appl. No.: 18/245,612

(22) PCT Filed: Sep. 29, 2021
(86) PCT No.: PCT/US2021/052648

§ 371 Date: Mar. 16, 2023

Related U.S. Application Data
(60) Provisional application No. 63/084,644, filed on Sep. 29, 2020.

(51) Int. Cl. C12Q 1/6886

(52) U.S. Cl. CPC C12Q 1/6886; C12Q 2600/118; C12Q 2600/158; C12Q 2600/106

(57) ABSTRACT

The present invention includes a method of determining a prognosis of a subject with cancer, and treatments thereof, comprising: obtaining or having obtained a sample from the subject; and measuring in the sample a level of expression of one or more Complement or Complement related genes or proteins; and determining if the levels of expression of the Complement or Complement related gene or protein when compared to the levels of expression of the Complement or Complement related genes or proteins from a subject that does not have cancer, wherein a change in the level of expression of the Complement or Complement related genes or proteins is associated with an unfavorable prognosis or a favorable prognosis.

### CROSS-REFERENCE TO RELATED APPLICATIONS

This application claims priority to U.S. Provisional Application Ser. No. 63/084,644, filed Sep. 29, 2020, the entire contents of which are incorporated herein by reference.

### STATEMENT OF FEDERALLY FUNDED RESEARCH

This invention was made with government support under R01CA190209 and P50CA196516 awarded by the National Institutes of Health. The government has certain rights in the invention.

### TECHNICAL FIELD OF THE INVENTION

The present invention relates in general to the field of biomarkers, and more particularly, to Complement as prognostic and predictive biomarker and potential therapeutic target in renal cell carcinoma.

### INCORPORATION-BY-REFERENCE OF MATERIALS FILED ON COMPACT DISC

The present application includes a Sequence Listing which has been submitted in ASCII format via EFS-Web and is hereby incorporated by reference in its entirety. Said ASCII copy, created on Sep. 28, 2021, is named TECH2153WO_ST25.txt and is 5, kilo bytes in size.

### BACKGROUND OF THE INVENTION

Without limiting the scope of the invention, its background is described in connection with renal cell carcinoma.

Early studies demonstrated deposition of complement proteins in several human malignancies [1]. Complement was thought to contribute to immune surveillance through complement dependent cytotoxicity (CDC) and tumor cell lysis [2]. These roles for complement are particularly clear in the context of anticancer monoclonal antibody therapies, where CDC assists in tumor cell killing, especially in hematologic malignancies [3]. However, because of overexpression of membrane and soluble complement regulatory (i.e. inhibitory) proteins, solid tumors are protected from complement-mediated lysis [2]. Consequently, the functional significance of complement activation in tumors in the absence of therapeutic antibodies has remained unclear.

In contrast to these beneficial antitumor complement roles, several studies demonstrated that the complement system promotes tumor growth by inhibiting antitumor immunity [4-6]. In fact, complement is currently perceived as an important immunosuppressive mechanism in primary tumors [6, 7] and metastasis-targeted organs [8, 9]. Complement proteins activate and recruit immunosuppressive cells, including myeloid-derived suppressor cells (MDSC), tumor-associated macrophages (TAM), and regulatory T cells (Tregs), to tumors and premetastatic niches [7, 10]. Recent work also demonstrated synergism between programmed cell death protein 1 (PD-1) blockade and complement inhibition to reduce tumor growth [11]. Interestingly, the C5a/C5a receptor 1 (C5aR1) axis was shown to have prognostic value in human renal cell carcinoma (RCC) [12]. In addition, the C1q and the classical pathway appear to contribute to RCC progression [13]. However, despite this foundational knowledge, complement-based anticancer therapies have not yet advanced to the clinic [14]. This may be due to two factors: (i) lack of understanding of best therapeutic targets within the complement cascade; and (ii) lack of knowledge about which cancer patients might benefit from complement-based therapies.

However, despite these advancements, a need remains for improved predictability and reliability of biomarkers, and methods of treatment for renal cell carcinomas.

### SUMMARY OF THE INVENTION

In one embodiment, the present invention includes a method of determining a prognosis of a subject with cancer comprising: obtaining or having obtained a sample from the subject; and measuring in the sample a level of expression of one or more Complement or Complement related genes or proteins; and determining if the levels of expression of the Complement or Complement related gene or protein when compared to the levels of expression of the Complement or Complement related genes or proteins from a subject that does not have cancer, wherein a change in the level of expression of the Complement or Complement related genes or proteins is associated with an unfavorable prognosis or a favorable prognosis. In one aspect, the cancer is selected from renal, urothelial, stomach, liver, pancreatic, breast, head/neck, testis, ovarian, and cervical. In another aspect, the Complement or Complement related gene or protein is selected from C1QA, C1QB, C1S, C1R, C2, C3, C5, C6, C7, CSB, CFB, CFD, CFH, CFI, CD21/CR2, CD46, CD55, CD59, C5AR1. In another aspect, the Complement or Complement related gene or protein is favorable and is selected from at least one of:

| Complement Gene | Cancer Type | Prognosis |
|---|---|---|
| C1S | Liver | Favorable |
| C3 | Liver | Favorable |
| C5 | Liver | Favorable |
| C6 | Liver | Favorable |
| C7 | liver | Favorable |
| C8B | Liver | Favorable |
| CFB | Breast | Favorable |
| CFD | Pancreatic | Favorable |
| CD21/CR2 | Breast | Favorable |
| CD46 | Stomach | Favorable |
| CD59 | Renal | Favorable |
| C5AR1 | Cervical | Favorable. |

In another aspect, the Complement or Complement related gene or protein is unfavorable and is selected from at least one of:

| Complement Gene | Cancer Type | Prognosis |
|---|---|---|
| C1QA | Renal | Unfavorable |
| C1QB | Renal | Unfavorable |
| C1S | Renal | Unfavorable |
| C1R | Renal | Unfavorable |
| C2 | Renal | Unfavorable |
| C3 | Renal | Unfavorable |
| CFB | Renal | Unfavorable |
| CFD | Renal | Unfavorable |
| CFH | Renal | Unfavorable |
| CFI | Urothelial | Unfavorable |
| CD46 | Cervical | Unfavorable |
| CD55 | Renal | Unfavorable |
| CD59 | Pancreatic | Unfavorable |
| | Head/Neck | Unfavorable |
| | Cervical | Unfavorable |
| C5AR1 | Renal | Unfavorable |
| | Testis | Unfavorable |
| | Ovarian | Unfavorable. |

In another aspect, a histological grade of the cancer is determined by the expressed or deposition of Complement proteins in tumor stroma. In another aspect, the Complement or Complement related gene or protein CFB, C5AR1, CFH, C3, C1R, CIS C1QA, and C1QB are enriched in aggressive inflammatory phenotype cancers. In another aspect, the method further comprises determining a level of expression of macrophage biomarkers selected from CD86, IRF1, STAB1; TFGB1, F13A1, IL-6, and CD40, wherein expression of one or more of the macrophage biomarkers is associated with an unfavorable prognosis. In another aspect, the sample is a plasma sample. In another aspect, the method further comprises separating a subject into a those with a higher or a lower level of expression of the Complement or Complement related gene or protein, and: if the subject has low FH and FD expression the subject has a worse response to an immune checkpoint inhibitor; if the subject has low FI and TCC the subject has a better response to an immune checkpoint inhibitor; or if the subject has low TCC and high C5 the subject has a better response to an immune checkpoint inhibitor. In another aspect, the immune checkpoint inhibitor is selected from nivolumab, ipilimumab, tremelimumab, ipilimumab and nivolumab, pembrolizumab, nivolumab, pidilizumab, MK-3475, MED 14736, CT-011, spartalizumab, durvalumab, atezolizumab, avelumab, AMP224, BMS-936559, MPLDL3280A, or MSB0010718C. In another aspect, the immune checkpoint inhibitor is selected from inhibitors of at least one of: CD137, CD134, PD-1, KIR, LAG-3, PD-L1, PDL2, CTLA-4, B7.1, B7.2, B7-DC, B7-H1, B7-H2, B7-H3, B7-H4, B7-H5, B7-H6, B7-H7, BTLA, LIGHT, HVEM, GAL9, TIM-3, TIGHT, VISTA, 2B4, CGEN-15049, CHK 1, CHK2, A2aR, TGF-beta, PI3Kgamma, GITR, ICOS, IDO, TLR, IL-2R, IL-10, PVRIG, CCRY, OX-40, CD160, CD20, CD52, CD47, CD73, CD27-CD70, or CD40. In another aspect, the method further comprises the step of treating a renal cell carcinoma with treated with C3aR1 and C5aR1 inhibitors to reduce tumor growth. In another aspect, the method further comprises the step of treating the subject with a complement blockade to at least one of: reduced vascular density in tumors or reduced expression of proangiogenic factors.

In another embodiment, the present invention includes a method of treating a subject with cancer comprising: obtaining or having obtained a sample from the subject; and measuring in the sample a level of expression of one or more Complement or Complement related genes or proteins; determining if the levels of expression of the Complement or Complement related gene or protein when compared to the levels of expression of the Complement or Complement related genes or proteins from a subject that does not have cancer, wherein a change in the level of expression of the Complement or Complement related genes or proteins is associated with an unfavorable prognosis or a favorable prognosis; and if the subject has low FH and FD expression the subject has a worse response to an immune checkpoint inhibitor; if the subject has low FI and TCC the subject has a better response to an immune checkpoint inhibitor; or if the subject has low TCC and high C5 the subject has a better response to an immune checkpoint inhibitor. In one aspect, the immune checkpoint inhibitor is selected from nivolumab, ipilimumab, tremelimumab, ipilimumab and nivolumab, pembrolizumab, nivolumab, pidilizumab, MK-3475, MED 14736, CT-011, spartalizumab, durvalumab, atezolizumab, avelumab, AMP224, BMS-936559, MPLDL3280A, or MSB0010718C. In another aspect, the immune checkpoint inhibitor is selected from inhibitors of at least one of: CD137, CD134, PD-1, KIR, LAG-3, PD-L1, PDL2, CTLA-4, B7.1, B7.2, B7-DC, B7-H1, B7-H2, B7-H3, B7-H4, B7-H5, B7-H6, B7-H7, BTLA, LIGHT, HVEM, GAL9, TIM-3, TIGHT, VISTA, 2B4, CGEN-15049, CHK 1, CHK2, A2aR, TGF-beta, PI3Kgamma, GITR, ICOS, IDO, TLR, IL-2R, IL-10, PVRIG, CCRY, OX-40, CD160, CD20, CD52, CD47, CD73, CD27-CD70, or CD40. In another aspect, the cancer is selected from renal, urothelial, stomach, liver, pancreatic, breast, head/neck, testis, ovarian, and cervical. In another aspect, a histological grade of the cancer is determined by the expressed or deposition of Complement proteins in tumor stroma. In another aspect, the Complement or Complement related gene or protein CFB, C5AR1, CFH, C3, C1R, CIS C1QA, and C1QB are enriched in aggressive inflammatory phenotype cancers. In another aspect, the method further comprises determining a level of expression of macrophage biomarkers selected from CD86, IRF1, STAB1, TFGB1, F13A1, IL-6, and CD40, wherein expression of one or more of the macrophage biomarkers is associated with an unfavorable prognosis. In another aspect, the sample is a plasma sample. In another aspect, the method further comprises the step of treating a renal cell carcinoma with treated with C3aR1 and C5aR1 inhibitors to reduce tumor growth. In another aspect, the method further comprises the step of treating the subject with a complement blockade to at least one of: reduced vascular density in tumors or reduced expression of proangiogenic factors.

In another embodiment, the present invention includes a method for treating a cancer comprising the steps of: performing or having performed a level of expression of one or more Complement or Complement related genes or proteins; determining if the levels of expression of the Complement or Complement related gene or protein when compared to the levels of expression of the Complement or Complement related genes or proteins from a subject that does not have cancer, wherein a change in the level of expression of the Complement or Complement related genes or proteins is associated with an unfavorable prognosis or a favorable prognosis; and if the subject has low FH and FD expression the subject has a worse response to an immune checkpoint inhibitor; if the subject has low FI and TCC the subject has a better response to an immune checkpoint inhibitor; or if the subject has low TCC and high C5 the subject has a better response to an immune checkpoint inhibitor. In one aspect, the immune checkpoint inhibitor is selected from nivolumab, ipilimumab, tremelimumab, ipilimumab and nivolumab, pembrolizumab, nivolumab, pidilizumab, MK-3475, MED 14736, CT-011, spartalizumab, durvalumab, atezolizumab, avelumab, AMP224, BMS-936559, MPLDL3280A, or MSB0010718C. In one aspect, the immune checkpoint inhibitor is selected from inhibitors of at least one of: CD137, CD134, PD-1, KIR, LAG-3, PD-L1, PDL2, CTLA-4, B7.1, B7.2, B7-DC, B7-H1, B7-H2, B7-H3, B7-H4, B7-H5, B7-H6, B7-H7, BTLA, LIGHT, HVEM, GAL9, TIM-3, TIGHT, VISTA, 2B4, CGEN-15049, CHK 1, CHK2, A2aR, TGF-beta, PI3Kgamma, GITR, ICOS, IDO, TLR, IL-2R, IL-10, PVRIG, CCRY, OX-40, CD160, CD20, CD52, CD47, CD73, CD27-CD70, or CD40.

### BRIEF DESCRIPTION OF THE DRAWINGS

For a more complete understanding of the features and advantages of the present invention, reference is now made to the detailed description of the invention along with the accompanying figures and in which:

FIGS. 1A to 1K shows the expression of complement genes in RCC and prognosis. Survival probabilities in patients with high vs. low expression of complement genes: (A) C1QA, cut off=197.13 Fragments Per Kilobase of Transcript per Million Mapped Reads (FPKM); (B) C1QB, cut off=184.01 FPKM; (C) CIS, cut off=37.42 FPKM; (D) C1R, cut off=38.62 FPKM; (E) C2, cut off=1.99 FPKM; (F) C3, cut off=97.97 FPKM; (G) C5AR1, cut off=4.36 FPKM; (H) CFB, cut off=4.86; (I) CFD, cut off=5.09 FPKM; (J) CFH, cut off=8.72 FPKM; (K) CD59, cut off=79.49 FPKM.

FIGS. 2A to 2S show the spatial distribution of complement proteins in RCC tumors and their association with grade. Detection of complement proteins in RCC tumors by immunohistochemistry: (A-E) C1qA; (F-G) C1qB; (H-L) C3; and (M-O) C5aR1; ccRCC-clear cell renal cell carcinoma, pRCC-papillary renal cell carcinoma; Stroma, Vasculature, Inf. Cells, and Tumor denote stromal deposition, vascular deposition or expression, expression in infiltrating or tumor cells, respectively; scale bars-50 μm, for A-0 n₁=22 males, average age 64±9.25 and n₂=24 females, average age 64.6±10.00; (P) C1qA expression by immunohistochemistry in low (grade 1-2) vs. high (grade 3-4) grade tumors; (Q) Semiquantitative analysis of C1qA expression as shown in P, in a scale 1-5, n_{1 low grade}=8, n_{2 high grade}=9, *P<0.0001 by t-test; (R) C3 expression by immunohistochemistry in low- v. high-grade tumors; and (S) Semiquantitative analysis of C3 expression as shown in C, n_{1low grade}=10, n_{2high grade}=13, *P<0.0001 by t-test.

FIGS. 3A to 3G shows the expression of complement genes in the inflammatory (IS) and non-inflammatory (NIS) subtypes of RCC. (A) Hierarchical clustering of the UTSW KCP RCC tumors based on the tumor microenvironment genes, which are the RCC-specific stroma/immune gene signature that the inventors previously defined. ssGSEA scores for immune cells, angiogenesis, and expression of complement genes (blue and green gene symbols for genes associated with unfavorable and favorable prognosis, respectively) are shown, with red in the graph meaning higher activation/expression scores. The RCC IS/NIS label, and the BAP1 and PBRM1 mutations are shown as green, blue, and black bars, respectively. The red bar denotes ccRCC (n=70) and white bar denotes all other RCC (n=111); Expression of: (B) complement, (C) chemokine, (D) T cell regulation & exhaustion, (E) myeloid cell regulation & function, and (F) enzyme genes in IS vs. NIS of the same patients as in A, *P<0.05, **P<0.01, ***P<0.001, and ****P<0.0001 by t-test, (G) Correlative analysis of complement genes' expression with genes associated with immunosuppression.

FIGS. 4A to 4R shows Complement proteins in RCC patients' plasma and the response to ICI. (4A-4F) Concentrations of complement proteins in plasma of RCC patients that were significantly different (P<0.05 by t-test) from concentrations in healthy donors; (4G-4J) P values of separation vs. threshold/cut off. The optimal threshold established is marked by blue triangle; (4K-4N) Treatment to next treatment time (TNT) in patients assigned to the cohorts based on cut offs shown in 4G-4J, (4K) P=0.022, (4L) P=0.029, (4N) P=0.017, (4M) P=0.042, all P values for K-M by t-test; (4O) Multifactor decision tree-algorithm to select patients with the low/high probability of responding to immune checkpoint inhibitors; (4P) Distribution of TNT: TNT<12 months-non-responders-red and TNT>12 months-responders-green; (4Q) C5 concentration and TCC, green dots outside the yellow gate denote responders with concentrations of C5>4.144 mg/dL and TCC<0.71973 AU/ml, the patient cohort inside this gate includes only three responders and sixteen non-responders-red crosses; (4R) TNT in multi-factor responders' cohort (shown outside a yellow gate of Q) vs. other patients, P=0.00000017 by t-test.

FIGS. 5A to 5G shows Complement in a mouse Renca model of RCC. (A) C3 deposition along CD31 antibody-stained vasculature; (B) C1q and C3; (C) C5a concentration in plasma from tumor-free (TF) and tumor-bearing (TB) mice, *P<0.0001 by t-test; (D) C1q and IgM; (E) Annexin V and IgM; (F) C5aR1 and CD11b; (G) C5aR1 and CD8f; Arrows denote areas of colocalization. Scale bars-50 am. (A-B) and (D-G) by immunofluorescence and (C) by ELISA.

FIGS. 6A to 6P show Complement inhibition in an anti-PD-1-recalcitrant model of RCC. Tumor volumes in: (A) isotype-IgG or anti-PD-1-treated mice; (B) placebo (PBS)-treated wild-type (WT), C3aR1 knockout (KO), or C5aR1 knockout (KO); and (C) placebo, SB290157 (C3aR1-inhibitor), or PMX53 (C5aR1-inhibitor)-treated mice (the same placebo cohort is shown in the panels B and C); (D-F) CD8⁺ T cells in tumors (TIL) from cohorts as in A, B, and C by FACS; (G-J) IFN-γ expressing TIL in cohorts as in A, B, and C: (G) representative FACS dot plots and (H-J) quantification of FACS data; (K) Expression of genes associated with T cell exhaustion in tumors from C3aR1KO, SB290157- and PMX53-treated mice relative to placebo (dashed line); (L) CD31 immunofluorescence (vascular density) in tumors from cohorts as in K and quantification of vascular density based on CD31 immunofluorescence; (M) Expression of proangiogenic genes in tumors form cohorts as in K relative to placebo (dashed line); (N-O) Representative FACS dot plots (with quantification) of spleen (N) and blood (0) from C3aR1KO and WT mice treated with anti-CD8α or isotype-matched IgG; (P) Tumor growth in cohorts as in N-O; ns-no significant, *P<0.05, **P<0.01 (in B-C3aR1 vs. placebo and in C-SB290157 or PMX53 vs. placebo),***P<0.001 (in P C3aR1-CD8⁺ T cells present vs. WT-CD8⁺ T cells present), and P****<0.0001 by Two-Way ANOVA for A-C and P, One-Way ANOVA for E, F, I, J, K, L, and M, and t-test for D, H, N and 0; data are representative of one experiment with n=5-15 mice.

FIGS. 7A to 7P shows the prognostic value of expression of genes upregulated in IS. Survival probabilities in patients with high vs. low expression of upregulated genes: (A) CCL4, cut off=3.14 Fragments Per Kilobase of Transcript per Million Mapped Reads (FPKM); (B) CCL5, cut off=29.03 FPKM; (C) CXCL10, cut off=3.17 FPKM; (D) CXCL11, cut off=0.21 FPKM; (E) SOCS1, cut off=2.74 FPKM; (F) PDCD1, cut off=0.88 FPKM; (G) CD27, cut off=2.68 FPKM; (H) CD86, cut off=5.8; (I) TRF1, cut off=5.03 FPKM; (J) STAB1, cut off=5.85 FPKM; (K) TGFB1, cut off=34.05 FPKM; (L) F13A1, cut off=16.05 FPKM; (M) IL6, cut off=0.66 FPKM; (N) CD40, cut off=20.02 FPKM; (0) TGM2, cut off=61.52 FPKM; (P) IDO1, cut off=0.74 FPKM. Based on the Human Protein Atlas.

FIGS. 8A to 8J show the concentrations of complement proteins in plasma collected with EDTA vs. heparin (CPT-tubes).

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The present inventors performed a systematic analysis of expression of several complement genes in human solid tumors to identify: (1) cancer patients with deregulated complement that might potentially benefit from complement-based interventions and (2) complement-dependent mechanisms regulating tumor growth that can be targeted for therapy. The inventors complemented these studies with comprehensive analyses of complement proteins in plasma and investigated their predictive potential for the response to immune checkpoints inhibitors (ICI). Findings in patients were corroborated in a mouse model of RCC.

The present inventors determined the best target for complement-based therapy amongst common human malignancies. High expression of eleven complement genes was linked to unfavorable prognosis in renal cell carcinoma. Complement protein expression or deposition was observed mainly in stroma, leukocytes, and tumor vasculature, corresponding to a role of complement in regulating the tumor microenvironment. Complement abundance in tumors correlated with a high nuclear grade. Complement genes clustered within an aggressive inflammatory subtype of renal cancer characterized by poor prognosis, markers of T cell dysfunction, and alternatively activated macrophages. Plasma levels of complement proteins correlated with response to immune checkpoint inhibitors. Corroborating human data, complement deficiencies and blockade reduced tumor growth by enhancing antitumor immunity and seemingly reducing angiogenesis in a mouse model of kidney cancer-resistant to PD-1 blockade. Thus, tumors resistant to immune checkpoint inhibitors are suitable targets for complement-based therapy. By identifying patients with the optimal biomarker profile of complement proteins who will respond to immune checkpoint inhibitors, healthcare providers will now be able to offer better survival outcomes, minimize adverse reactions and invasive procedures, and reduce costs for patients and insurance companies. As a result, present invention provides a less invasive, faster, and offers improved diagnostics for all patients.

Human Samples and Data

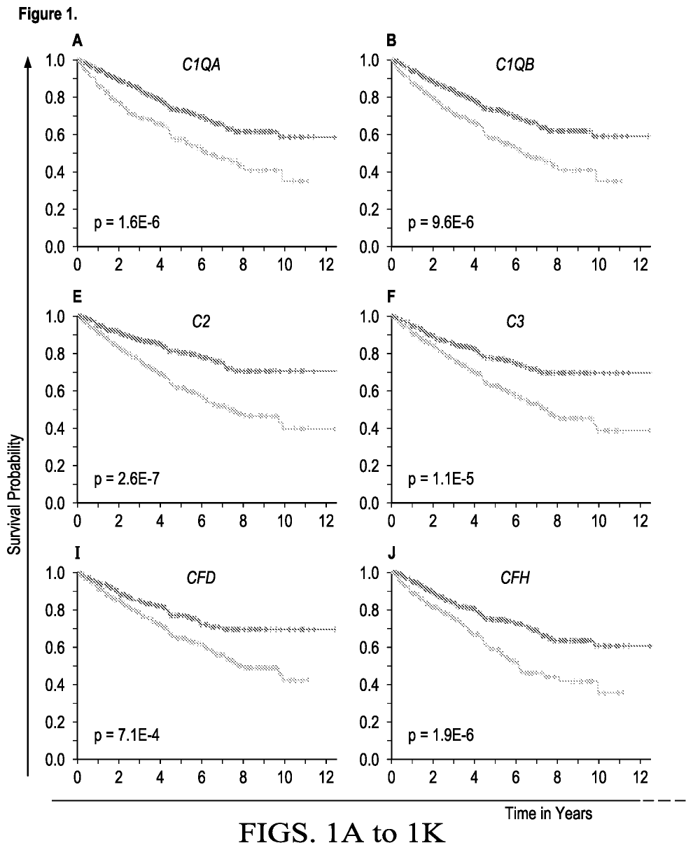

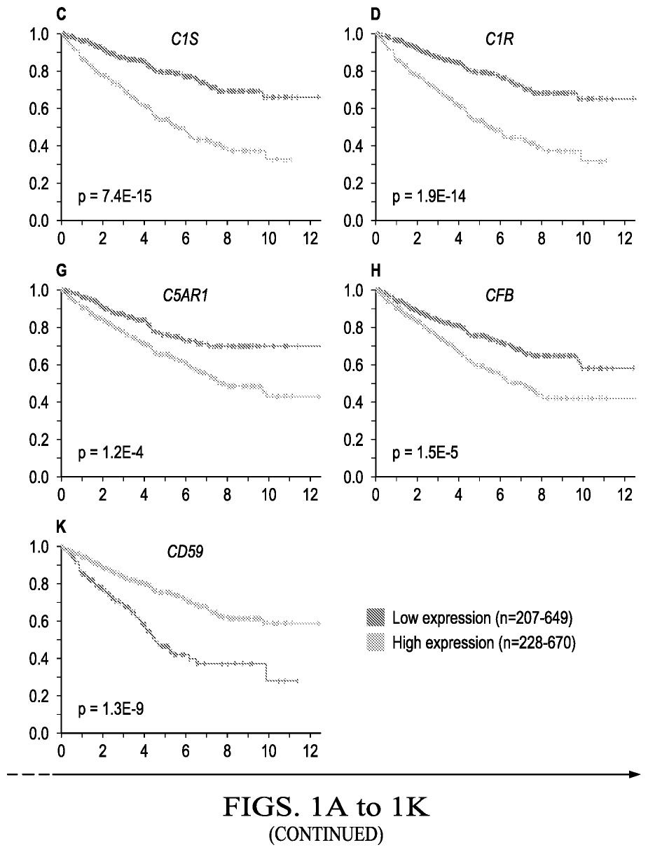

Data on complement genes' RNA expression and survival were obtained from Human Protein Atlas (https://www.proteinatlas.org/) and the Cancer Genome Atlas (https://cancergenome.nih.gov/) or from University of Texas Southwestern Medical Center Kidney Cancer Program (UTSW KCP) as previously reported [15]. The p values included in the Table I and FIG. 1 are based on the Kaplan-Meier survival analysis available through the Human Protein Atlas. Deidentified blood samples from RCC patients were obtained from UTSW KCP or from healthy donors from Oklahoma Blood Institute (Oklahoma City, OK). Blood from UTSW KCP was collected with ethylenediaminetetraacetic acid (EDTA) or with sodium heparin (CPT tubes) as anticoagulants. Blood from healthy donors was collected with EDTA. Blinded analysis of immunohistochemical staining of complement proteins in RCC and RCC grade, based on images available through Human Protein Atlas (https://www.proteinatlas.org/), was performed by a board-certified pathologist (MM). Scores from 1-5 were assigned based on semi-quantitative evaluation of intensity and pattern of staining and correlated with nuclear grade as established based on the International Society of Urological Pathology recommendations [16].

Data Availability

Sequencing data from UTSW KCP patients, specifically consenting to placement of their raw genomic data in a protected publicly accessible database, are deposited in the European Genome—phenome Archive (EGA) (https://www.ebi.ac.uk/ega/home), with accession numbers EGAS00001002786 and EGAS00001000926.

Mice, Cell Lines, and Treatments

Mouse studies were approved by the Institutional Animal Care and Use Committee of the Texas Tech University Health Sciences Center. Eight to twelve weeks old BALB/c, C3ar1, and C5ar1 knockout mice from the Jackson Laboratory were injected s.c. with 1×10⁶ Renca cells (ATCC® CRL2947™). When tumors reached ˜5 mm in diameter mice were assigned to treatment cohorts and treated with, C3aR1 inhibitor SB 290157 (Sigma-Aldrich, 10 mg/kg i.p. twice a day), C5aR1 inhibitor PMX53 [17] (1 mg/kg, i.p. every other day), or PD-1 antibody (RMP1-14, BE-0146, Bio X Cell, 250 μg per mouse i.p. every 4 days), or RaIgG2a isotype control (BE-0089, Bio X Cell, 250 μg i.p. every 4 days), as previously described [8, 18, 19]. CD8⁺ T cells were depleted by intraperitoneal (i.p.) injection of 200 μg of CD8a-neutralizing antibody (2.43, Bio X cell, NH) per mouse, each day, for three consecutive days, prior to injecting tumor cells. To maintain CD8⁺ T cell depletion, mice were injected with 200 μg of antibody every 3rd day beginning at day 3 after tumor cell injection. BALB/c control mice were treated in the same manner with rat IgG2b (LTF-2, Bio X cell).

Immunofluorescence

5-μm thick frozen tissue sections were stained with CD31 (Clone 390, BD Pharmingen), CD8α (53-6.7, BD Pharmingen), CD11b (550282, BD Biosciences), CD88(C5aR1) (sc-31240, Santa Cruz Biotechnology), C3b/iC3b/C3c, which binds only to C3 cleavage fragments but not to intact C3 [20] (HM 1065, Hycult Biotech), C1q (HM 1096BT, Hycult), mannan binding lectin (NB100-1502 Novus), IgM antibodies (14-5790-81, eBioscience) and Annexin V (sc-1929, Santa Cruz Biotechnology). Secondary antibodies included: Goat anti-rat antibodies (Invitrogen): Streptavidin-Cy2, Texas Red, and AF 633-conjugated; and donkey anti-goat AF 488-conjugated antibodies. Stainings were quantified with Nikon Elements Advanced Research Image-Analysis software based on analysis of at least ten fields per section. Data is expressed as the binary area occupied by positive cells.

Flow Cytometry

Cells from tumors were pre-incubated with CD16/32 antibody (Fc block; 2.4G2; BD Pharmingen) and stained with fluorochrome-conjugated antibodies from Biolegend: BV605-CD45 (30-F11), AF700-CD3 (17A2), PerCP/Cy5.5-CD4 (GK1.5), and PE/Cy7-CD8α (53-6.7) as recommended by the manufacturer. To quantify IFN-γ expression, cells were stained with surface markers and permeabilized with Cytofix/Cytoperm (554714, BD Biosciences), and washed with 1× Perm/Wash buffer (554714, BD Biosciences) followed by incubation with PE-IFN-γ (XMG1.2, Biolegend). Prior to intracellular staining, cells were incubated in the presence of brefeldin-A and monensin (BD Biosciences) in the presence of CD3 and CD28 antibodies (17A2 and 37.51, eBioscience) adsorbed to the 96-well plates for 6 to 8 h. Data were acquired on BD Fortessa and analyzed with FlowJo software (Tree Star).

### Real Time Quantitative PCR

RNA was extracted from frozen tissue using RNeasy plus mini kit (Qiagen) and cDNA was generated using High-Capacity RNA-to-cDNA™ kit (Applied Biosystems). The q(RT)-PCR was performed using High Capacity cDNA Synthesis Kit, Fast SybrGreen, StepOnePlus Applied Biosystems. Relative expression was calculated using the 2^{-ΔΔCt} method and RT2 profiler PCR Array Data Analysis (SAB Biosciences) and normalized to GAPDH. The primer sequences are as follows:

| | Vegfa: |
|---|---|
| (SEQ ID NO: 1) | |
| | 5′-CTGCTGTAACGATGAAGCCCTG-3′ |
| | and |
| (SEQ ID NO: 2) | |
| | 5′-GCTGTAGGAAGCTCATCTCTCC-3′; |
| | Vegfb: |
| (SEQ ID NO: 3) | |
| | 5′-ACTGGGCAACACCAAGTCCGAA-3′ |
| | and |
| (SEQ ID NO: 4) | |
| | 5′-CACATTGGCTGTGTTCTTCCAGG-3′; |
| | Vegfc: |
| (SEQ ID NO: 5) | |
| | 5′-CCTGAATCCTGGGAAATGTGCC-3′ |
| | and |
| (SEQ ID NO: 6) | |
| | 5′-CGATTCGCACACGGTCTTCTGT-3′; |
| | Angpt1: |
| (SEQ ID NO: 7) | |
| | 5′-AACCGAGCCTACTCACAGTACG-3′ |
| | and |
| (SEQ ID NO: 8) | |
| | 5′-GCATCCTTCGTGCTGAAATCGG-3′; |
| | Tek: |
| (SEQ ID NO: 9) | |
| | 5′-GAACTGAGGACGCTTCCACATTC-3′ |
| | and |
| (SEQ ID NO: 10) | |
| | 5′-TCAGAAACGCCAACAGCACGGT-3′; |
| | Illb: |
| (SEQ ID NO: 11) | |
| | 5′-TGGACCTTCCAGGATGAGGACA-3′ |
| | and |
| (SEQ ID NO: 12) | |
| | 5′-GTTCATCTCGGAGCCTGTAGTG-3′; |
| | Ctla4: |
| (SEQ ID NO: 13) | |
| | 5′-GTACCTCTGCAAGGTGGAACTC-3′ |
| | and |
| (SEQ ID NO: 14) | |
| | 5′-CCAAAGGAGGAAGTCAGAATCCG-3′; |
| | Pdcd1: |
| (SEQ ID NO: 15) | |
| | 5′-ACCCTGGTCATTCACTTGGG-3′ |
| | and |
| (SEQ ID NO: 16) | |
| | 5′-CATTTGCTCCCTCTGACACTG-3′; |
| | Btla: |
| (SEQ ID NO: 17) | |
| | 5′-CTTCTGCTCCTTGCCTGTGTCT-3′ |
| | and |
| (SEQ ID NO: 18) | |
| | 5′-GGTTAGTGTCCCTTCCTGCCAA-3′; |
| | Fas: |
| (SEQ ID NO: 19) | |
| | 5′-CTGCGATTCTCCTGGCTGTGAA-3′ |
| | and |
| | Stat3: |
| (SEQ ID NO: 20) | |
| | 5′-CAACAACCATAGGCGATTTCTGG-3′; |
| (SEQ ID NO: 21) | |
| | 5′-AGGAGTCTAACAACGGCAGCCT-3′ |
| | and |
| (SEQ ID NO: 22) | |
| | 5′-GTGGTACACCTCAGTCTCGAAG-3′. |

ELISA of Human and Mouse Plasma

Mouse C5a ELISA was performed according to the manufacturer's instructions (DY2150, R&D). Human complement ELISA kits C1q (HK356-02), C3 (HK366-02), C5 (HK390-02), factor B, (FB, HK367-02) factor H (FH, HK342-02), factor D (FD, HK-343-02), factor I (FI, HK355-02), C3c (HK-368). sCD59 (HK374-02), and soluble (s)C5b-9, known as membrane attack complex (MAC) or complement terminal complex (TCC) (HK328-01) were obtained from Hycult Biotech (Uden, Netherlands) and were used according to the manufacturer's recommendations.

Statistics

Data were analyzed with t-test or One-way ANOVA (more than two mean values comparison). Impact of treatments on growth of mouse tumors over time was evaluated by Two-way ANOVA. The log-rank test was used for patient survival analysis and survival data were visualized by the Kaplan-Meier estimator (The Human Protein Atlas). To determine the predictive value of complement proteins for the response to ICI the inventors used the time to next treatment (TNT) as a surrogate of response. The inventors divided patients into cohorts with protein concentration above or below a set threshold. Then, the inventors compared the distribution of TNT and calculated the p-value of a difference between the cohorts. The optimum threshold was set at the minimum p-value of separation, corresponding to a maximal difference in TNT. Multiple-protein analysis used the scikit-learn machine learning library version 0.22 [21]. The p<0.05 were considered significant. Bar graphs indicate mean±SEM. GraphPad Prism 6 was used for analyses.

High expression of complement genes is associated with unfavorable prognosis in RCC.

Using data available through the Human Protein Atlas (https://www.proteinatlas.org/) and the Cancer Genome Atlas (https://cancergenome.nih.gov/) the inventors analyzed expression of complement genes in human solid tumors (Table I). The inventors found eleven soluble complement proteins, receptors, and regulators that were associated with poor prognosis in RCC (Table I and FIG. 1A-J [10 genes are shown]). One complement protein, CD59, was associated with improved outcomes (FIG. 1K), but CD59 is a negative regulator of the complement system, also known as C5b-9/MAC/TCC-inhibitory protein [22]. In contrast to RCC, several complement genes were linked to favorable prognosis in other common human tumors including, liver, pancreatic, breast, and cervical carcinomas (Table I).

TABLE I
Complement genes and Prognosis in Human Cancers.

| Complement Gene | Cancer Type | Prognosis | p-value |
|---|---|---|---|
| C1QA | Renal | Unfavorable | 1.58E−06 |
| C1QB | Renal | Unfavorable | 9.58E−06 |
| C1S | Renal | Unfavorable | 7.44E−15 |
| | Liver | Favorable | 7.73E−04 |
| C1R | Renal | Unfavorable | 1.94E−14 |
| C2 | Renal | Unfavorable | 2.64E−07 |
| C3 | Renal | Unfavorable | 1.09E−05 |
| | Liver | Favorable | 8.11E−04 |
| C5 | Liver | Favorable | 9.43E−04 |
| C6 | Liver | Favorable | 4.31E−04 |
| C7 | liver | Favorable | 5.89E−04 |
| C8B | Liver | Favorable | 9.30E−05 |
| CFB | Renal | Unfavorable | 1.53E−05 |
| | Breast | Favorable | 2.76E−05 |
| CFD | Pancreatic | Favorable | 1.77E−04 |
| | Renal | Unfavorable | 7.09E−04 |
| CFH | Renal | Unfavorable | 1.92E−06 |
| CFI | Urothelial | Unfavorable | 6.06E−04 |
| CD21/CR2 | Breast | Favorable | 6.90E−04 |
| CD46 | Cervical | Unfavorable | 8.54E−05 |
| | Stomach | Favorable | 2.38E−04 |
| CD55 | Renal | Unfavorable | 9.96E−04 |
| CD59 | Renal | Favorable | 1.30E−09 |
| | Pancreatic | Unfavorable | 2.92E−05 |
| | Head/Neck | Unfavorable | 3.10E−05 |
| | Cervical | Unfavorable | 3.81E−05 |
| C5AR1 | Renal | Unfavorable | 1.16E−04 |
| | Testis | Unfavorable | 7.99E−04 |
| | Ovarian | Unfavorable | 9.55E−04 |
| | Cervical | Favorable | 4.71E−04 |

Complement proteins are expressed or deposited in tumor stroma and their abundance correlate with histological grade

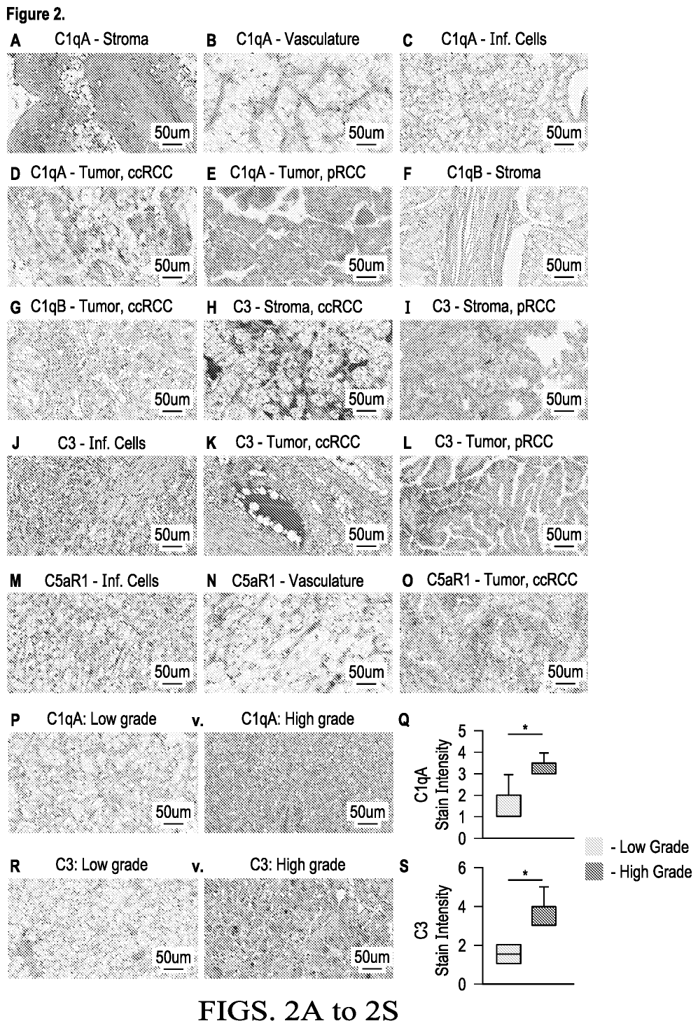

Tumor-promoting functions of complement proteins are linked to the immunosuppressive tumor microenvironment (TME) [23]. However, some studies demonstrated that complement promotes tumor growth via direct autocrine effect on tumor cells that is independent from inhibiting antitumor T cells [24]. Therefore, it is important to determine localization of complement proteins in tumors. The inventors focused the analyses on C1qA (n=22), C1qB (n=11), C3 (n=29), and C5aR1 (n=22) because of the prognostic value of these proteins in RCC, their strategic positions in the complement cascade, and their roles in regulating tumor growth in mouse models [6]. Immunohistochemistry slides (84 samples) from 43 RCC patients, available through the Human Protein Atlas, were analyzed. C1qA was present as extracellular deposits in stroma (FIG. 2A), or was associated with the vasculature (FIG. 2B), and/or infiltrating cells/leukocytes (FIG. 2C) in all 22 tumor samples. Of 9 samples with cytoplasmic staining of C1qA in tumor cells, 6 were clear cell (cc) RCC and 3 were papillary (p) RCC (FIG. 2D, E). C1qB staining was limited to stromal deposits, scarce infiltrating cells, and vasculature (FIG. 2F) in 10 out of 11 ccRCC samples. The weak and focal staining of tumor cells was found only in one high-grade ccRCC (FIG. 2G). The most consistent C3 staining pattern was stromal and vascular deposition in all 29 samples, regardless of histologic subtype (FIGS. 2H and 2I). C3 positive infiltrating cells were observed in 10 of 29 tumor samples (FIG. 2J). C3 cytoplasmic and membrane staining of tumor cells was observed in 12 out of 24 ccRCC sections (FIG. 2K) and in 2 out of 5 pRCC sections (FIG. 2L). C5aR1 expression was limited to infiltrating cells (FIG. 2M) and the vasculature (FIG. 2N) in 17 out of 22 ccRCC samples. Membrane staining of tumor cells was observed in only 5 high-grade ccRCC (FIG. 2O). A semi-quantitative analysis of C1qA and C3 staining demonstrated that high-grade tumors (nuclear grade 3-4) had more widespread staining and higher intensity than low-grade (1-2) tumors (FIG. 2 P-S).

Complement genes are enriched in an aggressive inflammatory subtype of RCC

The inventors previously reported the discovery of an inflammatory subtype (IS) of RCC characterized by local immune cell infiltration, systemic inflammation, poor prognosis, and BAP1 mutations [15]. In that report, the inventors used publicly available RNA-sequencing data sets from TCGA (n=529 ccRCC) as well as from UTSW KCP (n=181, including 39%, 24%, 15%, and 22% patients with ccRCC, pRCC, chromophobe, and other tumor-types, respectively). The IS cluster was enriched for gene signatures of T regulatory cells (Tregs), natural killer (NK) cells, Th1 cells, neutrophils, macrophages, B cells, CD8⁺ T cells, and C1q [15]. The identification of CD8⁺ T cells (tumor infiltrating lymphocytes-TIL) in this cluster was not surprising as CD8⁺ T cells have been previously associated with poor prognosis in RCC, unlike in other tumor types [25].

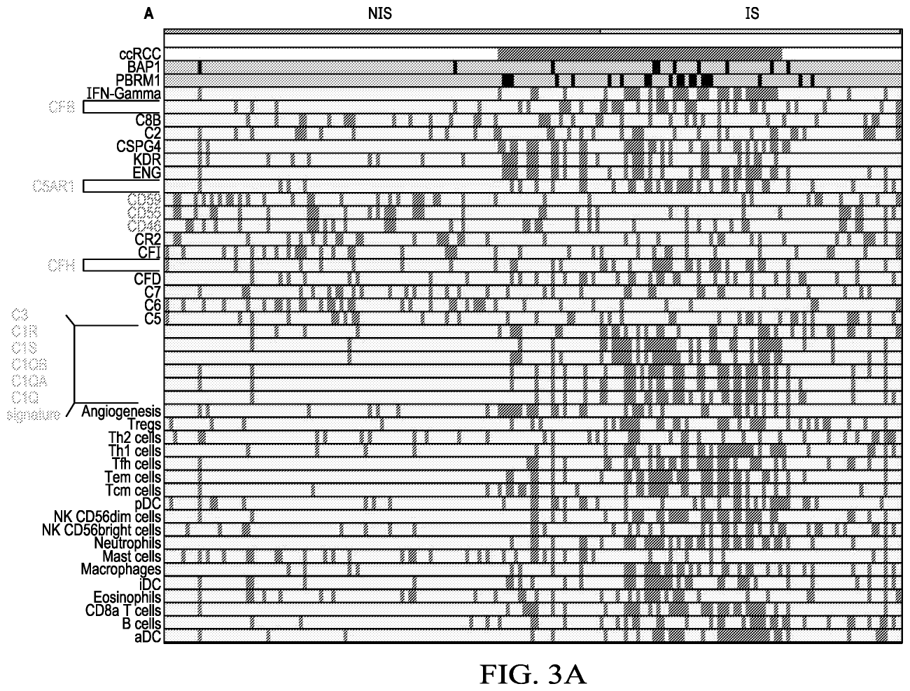

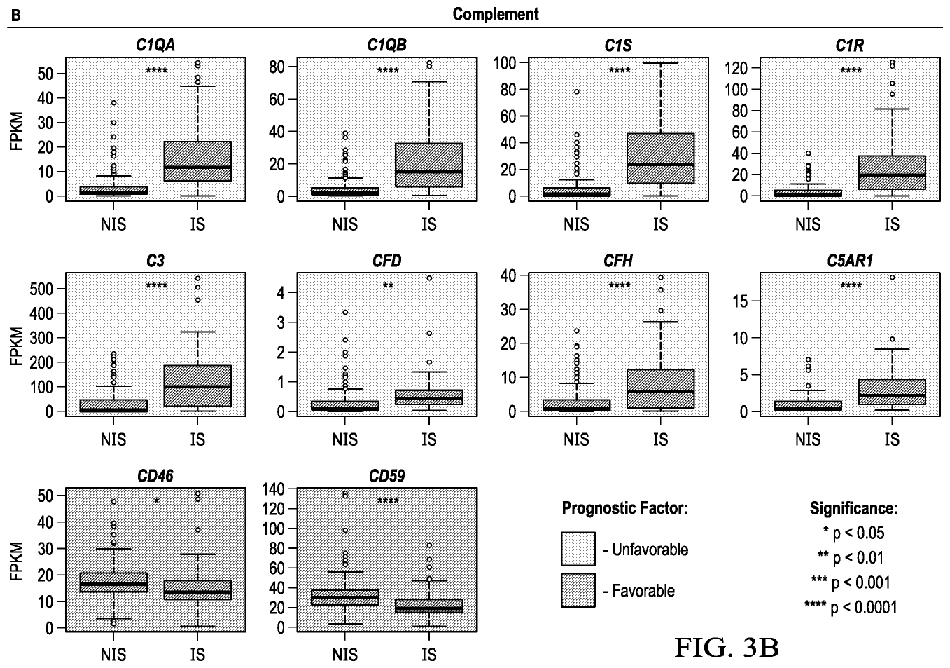

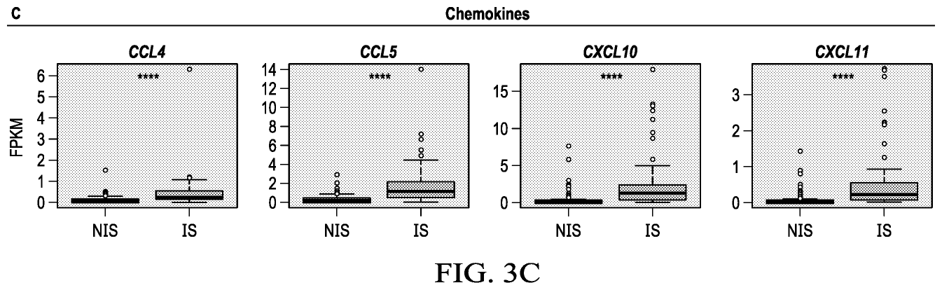

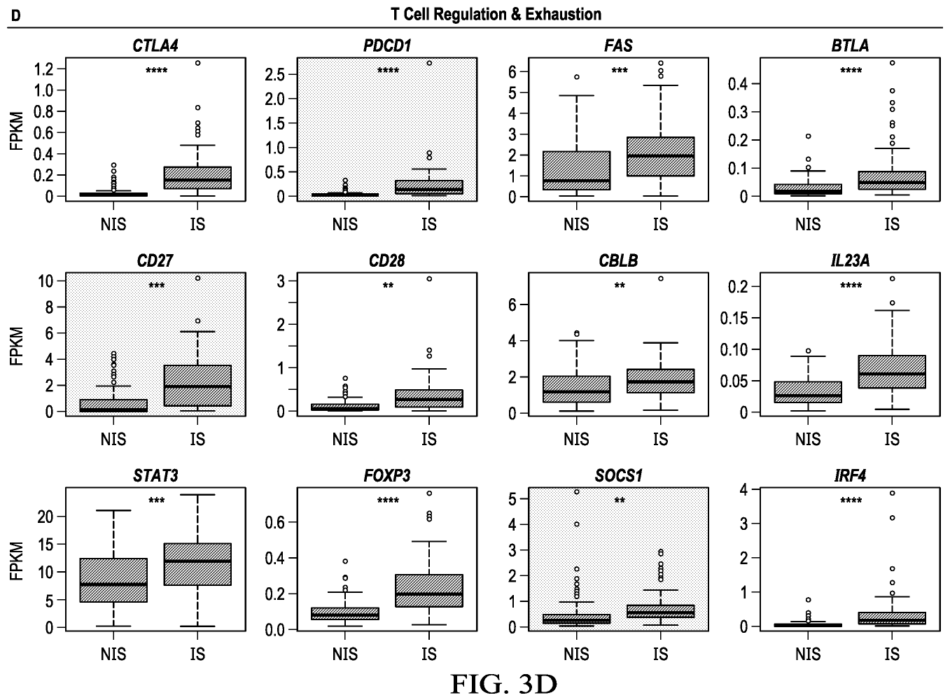

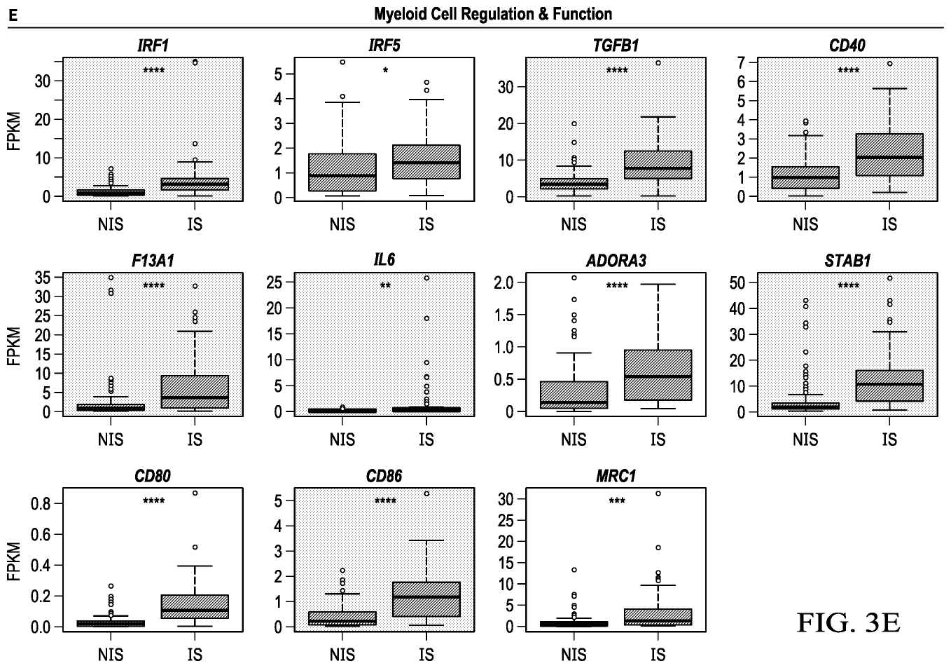

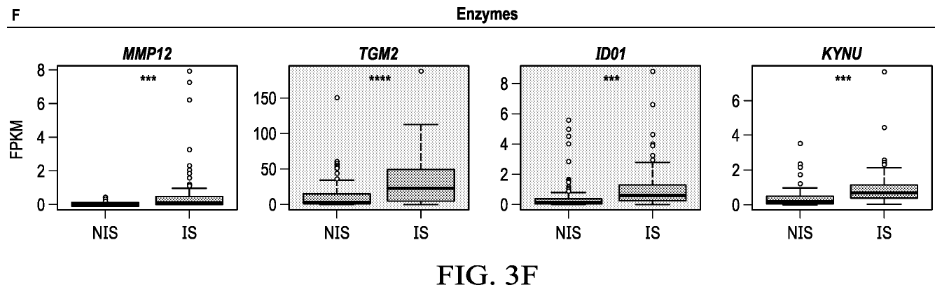

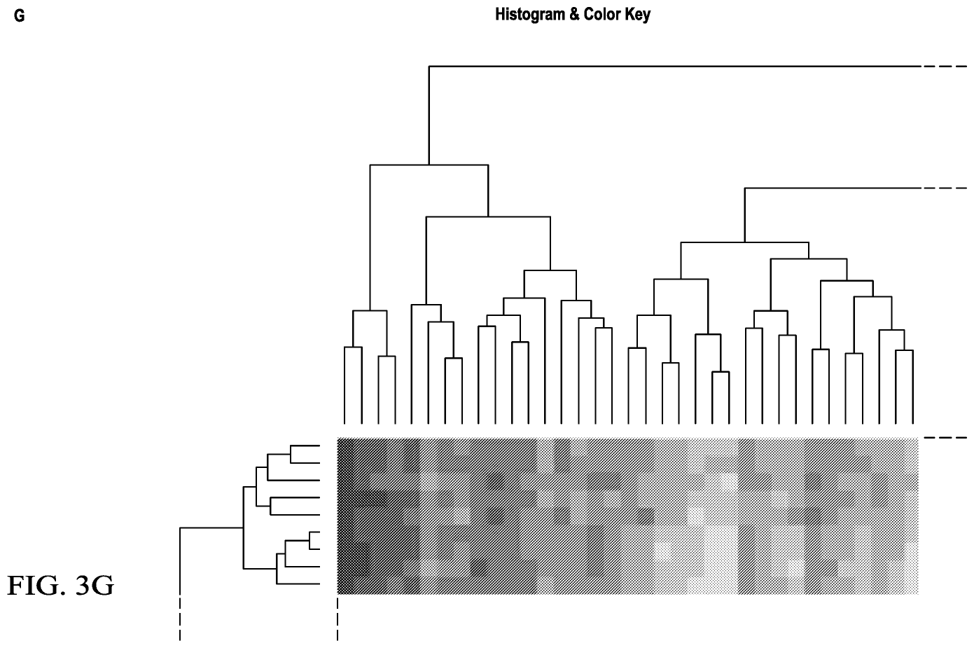

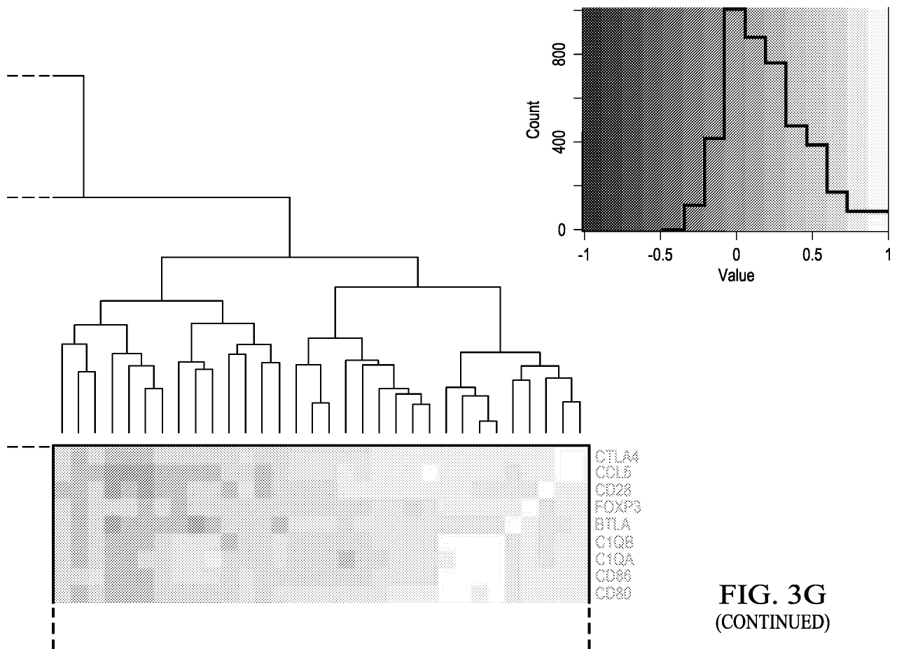

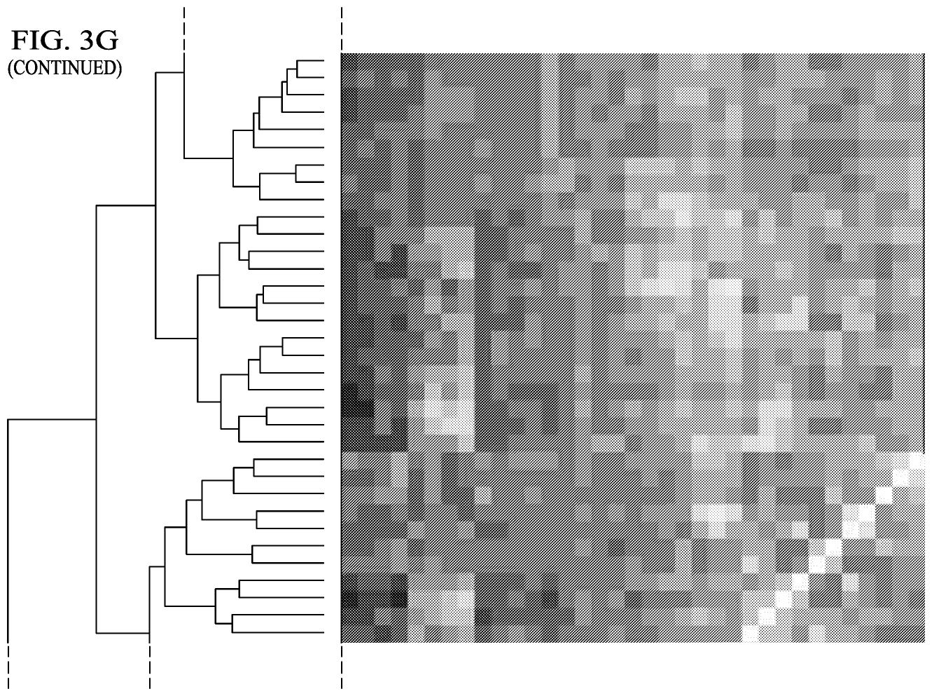

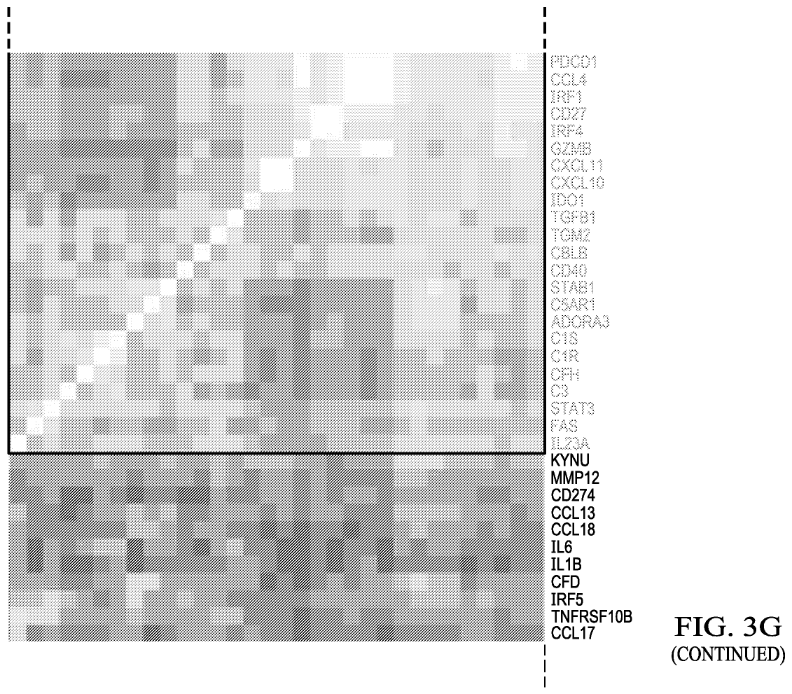

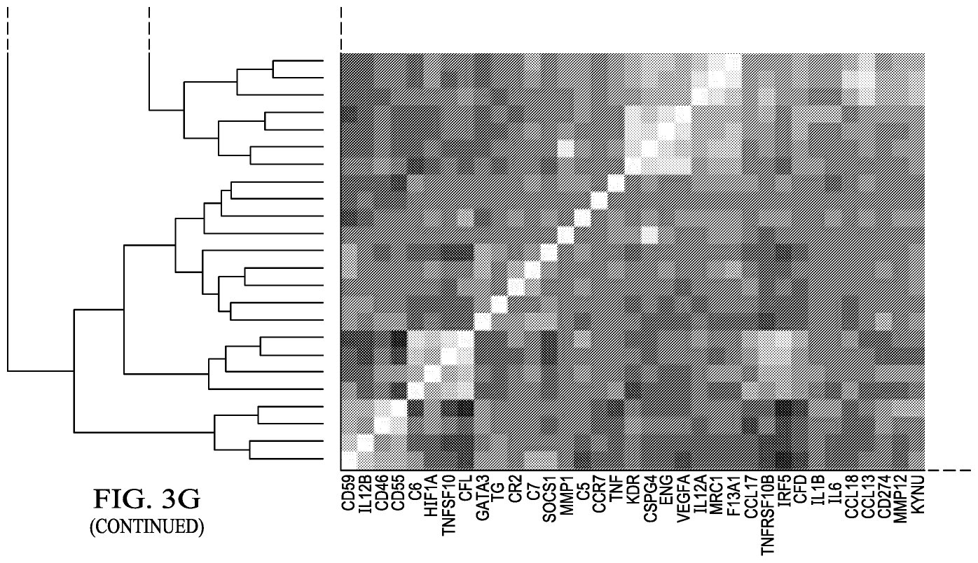

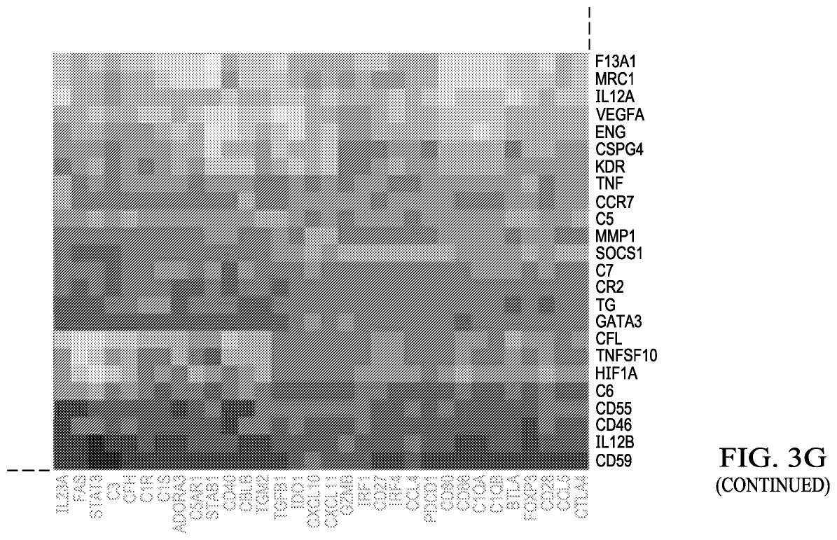

The inventors reanalyzed the UTSW KCP data for complement-related genes. CFB, C5AR1, CFH, C3, CIR, CIS C1QA, and C1QB were enriched in the IS, in comparison with the non-IS subtype (NIS), especially for ccRCC patients (FIG. 3A, blue gene symbols in boxes). Conversely, complement regulatory genes (CD46, CD55, and CD59) encoding proteins inhibiting/controlling complement activation were enriched in NIS RCC (FIG. 3A-green gene symbols). These data extend the findings in FIG. 1, by showing that complement protein expression is associated with an IS of RCC, which the inventors previously showed is characterized by poor prognosis [15].

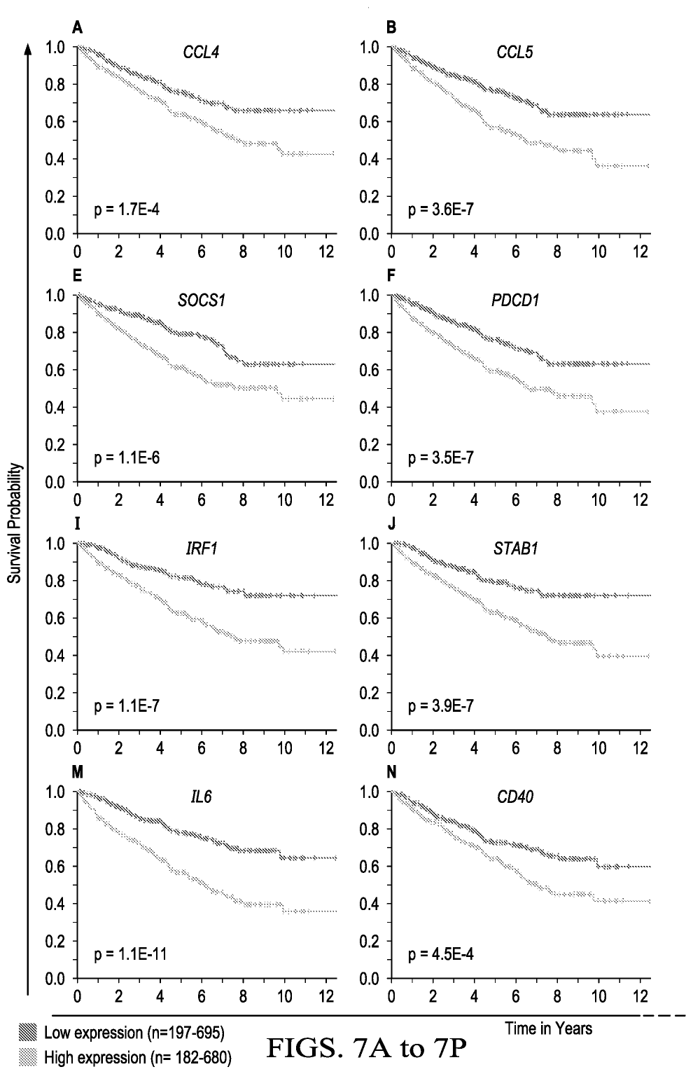

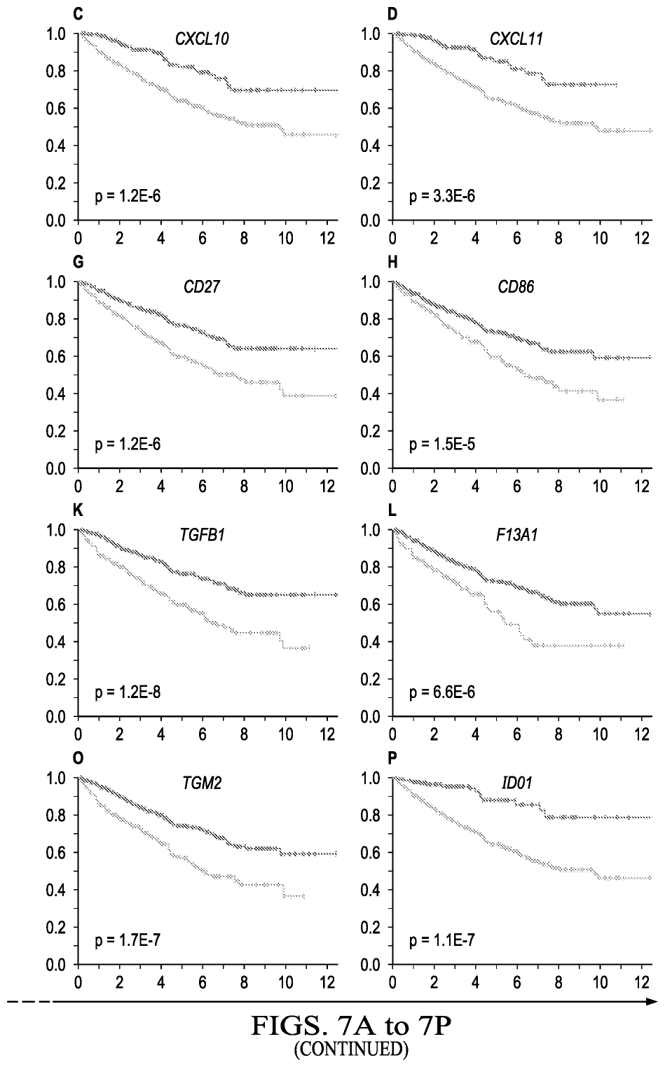

The direct comparison of complement genes in IS vs. NIS demonstrated higher expression of genes associated with poor prognosis in IS. In contrast, CD59, which is associated with favorable prognosis, had high expression in NIS together with another complement regulator CD46 (FIG. 3B). In addition, several chemokine encoding genes had relatively higher expression in the IS vs. NIS (FIG. 3C). These chemokines are implicated in regulating growth of several cancers, recruiting immune cells to tumors, and their expression is associated with T cell exhaustion [26, 27]. Importantly, high expression of CCL4, CCL5, CXCL10, and CXCL11 was associated with worse outcomes in RCC (FIGS. 7A-D).

Poor prognosis associated with TIL in RCC and high number of TIL in IS suggests that these T cells are dysfunctional. The inventors evaluated genes associated with T cell exhaustion/dysfunction [28] in IS vs. NIS and found that they upregulated in IS (FIG. 3D). Several of these genes (highlighted by pink background in FIG. 3D) were also associated with reduced patient survival (FIGS. 7E-G). Because macrophage genes were enriched in IS [15] and TAM and other cells of myeloid origin in tumors are immunosuppressive [29], the inventors evaluated genes linked to myeloid cell regulation and function. Several alternatively activated macrophage markers and macrophage regulators [30] had higher expression in IS vs. NIS (FIG. 3E). Among those, high expression of CD86, IRF1, STAB1, TFGB1, F13A1, IL-6, and CD40 was associated with an unfavorable prognosis (FIGS. 7H-N). Genes encoding enzymes involved in extracellular matrix remodeling and immunosuppression also had higher expression in IS vs. NIS (FIG. 3F). In this category, TGM2 and IDO1 were associated with lower survival (FIGS. 70-P). Importantly, several genes potentially involved in suppression of antitumor immunity in RCC in the FIG. 3C-F positively correlated with complement genes (FIG. 3G-gated area), suggesting that complement pathways may be intertwined with immunosuppressive mechanisms supporting immune escape of tumor cells in RCC.

Complement in Plasma and the Response to Immune Checkpoint Inhibitors

In general, the contribution of non-PD-1/CTLA-4 pathways to tumor immunosuppression predicts lack of or limited response to ICI [31]. Because the high expression of complement genes is associated with: 1) T cell exhaustion, and 2) activated macrophage markers, the inventors hypothesized that complement may contribute to these immunosuppressive pathways and that increased complement activity may predict resistance to ICI. Several complement proteins are secreted from cells and circulate in plasma. Complement function is routinely evaluated in plasma in the clinic [32]. Furthermore, the inventors previously showed in mouse models that activation of complement in tumors or in metastatic sites is reflected by changes in concentrations of complement effectors in plasma [8]. Thus, the inventors evaluated complement function in plasma and sought to establish whether plasma levels of complement proteins was associated with response to ICI.

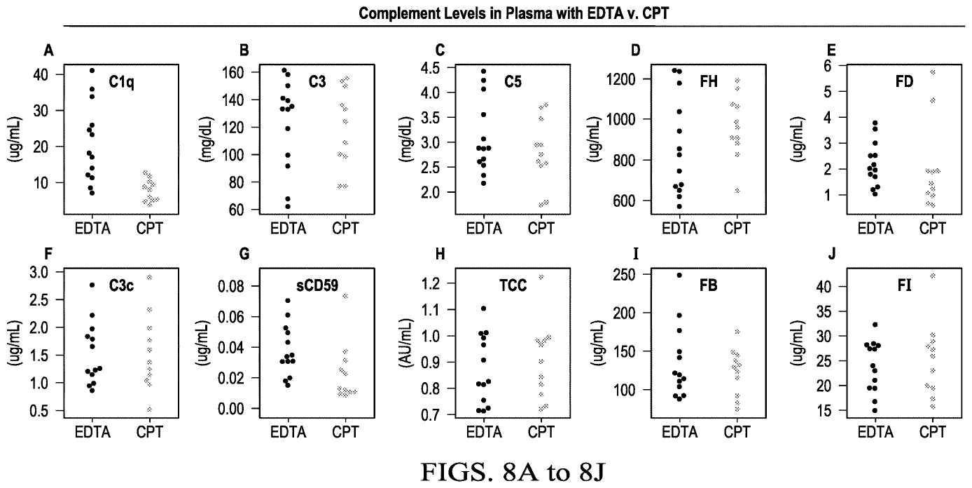

The inventors measured the concentrations of C1q, C3, C5, FB, FD, FH, FI, C3c, sCD59, and s5b-9 (TCC) in plasma collected from 24 RCC patients treated at the UTSW KCP prior to initiation of ICI (nivolumab monotherapy (n=19) or combination ipilimumab and nivolumab (n=5)) (Table 2) and from healthy donors (Oklahoma Blood Institute). The concentration of plasma complement proteins were correlated with time to next treatment (TNT), a surrogate of response to ICI. TNT is defined as a time from starting of ICI until the next line of therapy, which is usually administrated because of disease progression. The inventors used TNT as it captures patient benefit from ICI beyond the treatment course, and it is less subjective than retrospective interpretation of imaging studies, which is also influenced by the criteria used and pseudoprogression. Thus, a shorter TNT indicates a limited response to therapy. For measurements of complement proteins, the inventors used commercially available ELISA kits that are designed to work with plasma collected with different anticoagulants. However, as with other laboratory assays, the sample collection method may impact results. Therefore, the inventors sought to determine data distribution in samples collected with EDTA vs. heparin (CPT) because the analysis includes plasma collected with both anticoagulants. For all measured complement fragments, except C1q, data distributions were similar (FIGS. 8A to 8J), indicating that the sample collection was unlikely to affect the outcomes of these assays. C1q concentration was affected by heparin (FIG. 8A), therefore, only EDTA C1q samples were included in the subsequent analyses.

TABLE 2

| Demographics (n = 24) | | |
|---|---|---|
| Mean Age at diagnosis (years) | 59.96 ± 11.03 | |
| Sex | | |
| Male | 20 | (83.34%) |
| Female | 4 | (16.67%) |
| Clinical Characteristics | | |
| Histology | | |
| ccRCC | 22 | (91.68%) |
| pRCC (HLRCC) | 1 | (4.16%) |
| Chromophobe RCC | 1 | (4.16%) |
| Grade | | |
| Low (1-2) | 5 | (20.84%) |
| High (3-4) | 18 | (75.00%) |
| N/A | 1 | (4.16%) |
| Treatment Type | | |
| Nivolumab | 19 | (79.17%) |
| Nivolumab + Ipilimumab | 5 | (20.83%) |

| | |
|---|---|
| Length of Treatment (months) | 2.3-25.1 |

### The RCC UTSW Medical Center Cohort for Blood Analyses.

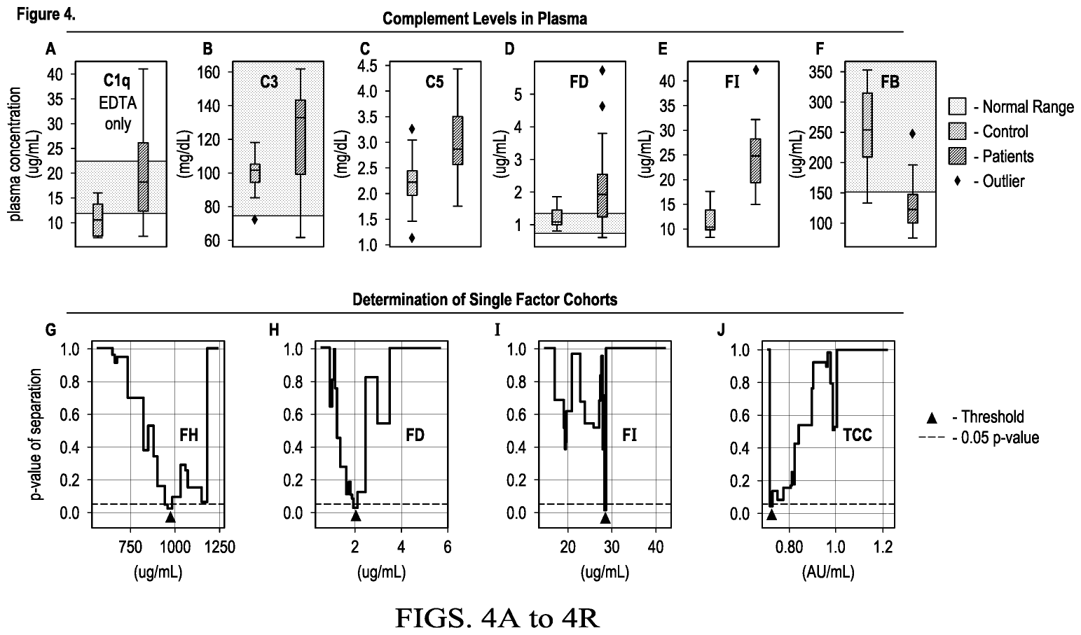

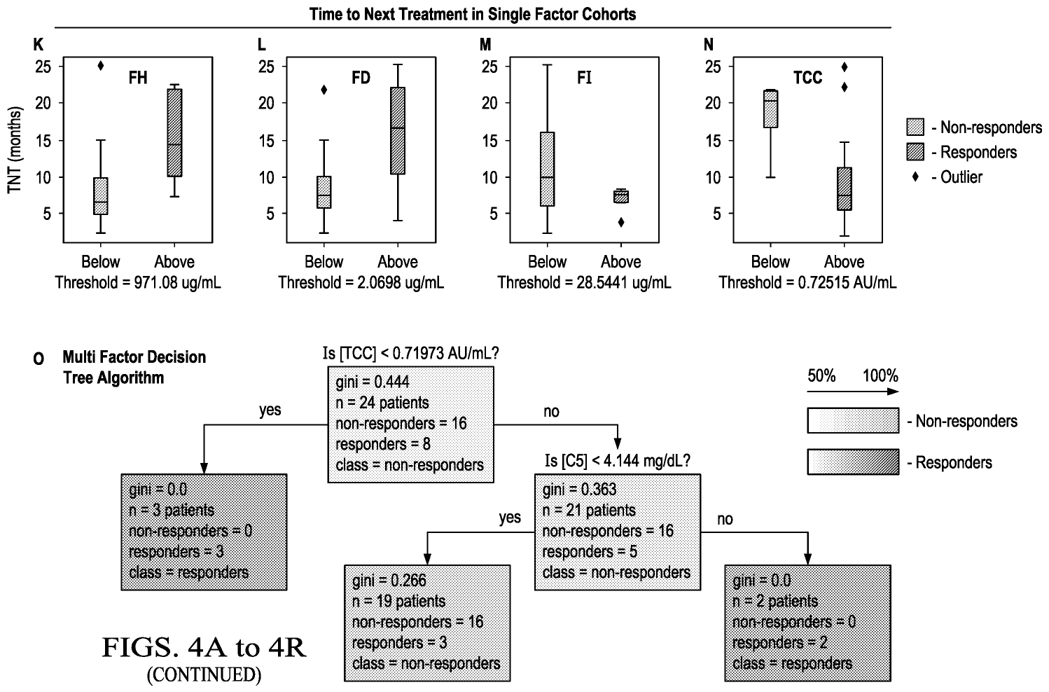

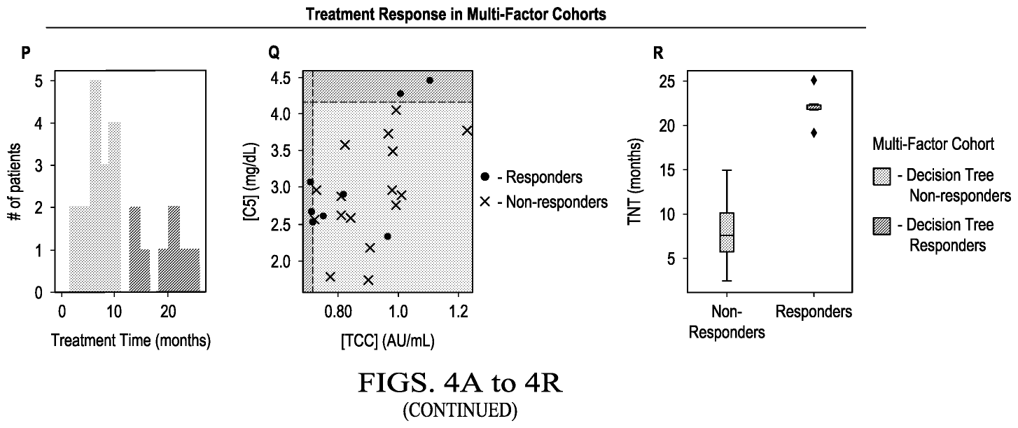

The inventors found that the concentration of several complement proteins in RCC patients' plasma was significantly higher than in control plasma from healthy donors except for FB. (FIGS. 4A-4F). C1q and C3 in most of RCC patients were within what is regarded as the normal range (FIG. 4A, 4B). For C5 and FI references ranges varies significantly between laboratories, therefore, they are not included in this figure. FI and FB in patients were higher or lower than in controls, respectively (FIGS. 4E, 4F). Moreover, FB levels in RCC patients were below normal range (FIG. 4F). To separate patients into cohorts with high vs. low concentrations of complement proteins the inventors used threshold (cut off), established based on the minimum (lowest) p-value (p-value of separation), corresponding to a maximum difference in TNT (FIGS. 4G-4J). The patients with low FH and FD (below cut-off) had worse response to ICI indicated by significantly shorter TNT (FIGS. 4K, 4L). Conversely, low FI and TCC were associated with better response indicated by significantly longer TNT (FIGS. 4M, 4N).

Next, the inventors sought to determine if using plasma concentrations of several complement proteins simultaneously would provide better prediction of response to ICI than the concentration of any single protein. To identify which combination of complement protein concentrations will be the best predictor, the inventors used the scikit-learn machine learning library [21] (http://scikit-learn.org), to establish an optimal decision tree (FIG. 4O). Based on the distribution of TNT (FIG. 4P), patients were separated into two cohorts using 12 months as the TNT cutoff (TNT<12 months-red color code, worse responders & TNT>12 months-green color code, better responders). The inventors trained a decision tree using C1q, C3, C5, FB, FD, FH, FI, C3c, sCD59, and s5b-9 (TCC) concentrations as input features to find optimal separation with constrained tree depth. Decision trees are “grown” iteratively by finding the single variable split that best subdivides the group into cohorts (as measured by the greatest decrease in the node's Gini impurity) [33]. The algorithm searched through all proteins looking for a single split that best subdivided all patients (n=24). The optimal move was to separate patients based on whether TCC is above or below 0.71973AU/ml (FIG. 4O), as three patients with concentrations below this threshold were responders (FIG. 4O). The algorithm was repeated on the remaining patients (n=21) determining that a C5 concentration threshold of 4.144 mg/dL optimally split this group as two patients with concentrations of C5>4.144 mg/dL and TCC<0.71973AU/ml were responders (FIG. 4O). To reduce chances of data overfitting, the inventors stopped the algorithm at this point. Thus, of the original patients (n=24), the decision tree identified a cohort of five patients with C5>4.144 mg/dL and TCC<0.71973AU/ml that were all responders (FIG. 4Q, green dots outside the yellow gate). The average TNT in this group was significantly higher than in the alternative group containing 16 non-responders and 3 responders (FIG. 4Q-red crosses and green dots in a yellow gate). Thus, highest benefit from ICI is observed among patients with low TCC and high C5. To extend these conclusions to the general RCC patient population, larger patient cohorts will be required. FIG. 4R shows TNT in multi-factor responders' cohort (shown outside a yellow gate of Q) vs. other patients, P=0.00000017 by t-test.

Complement inhibition reduces growth of anti-PD-1-resistant renal tumors in mice by improving TIL function and inhibits angiogenesis

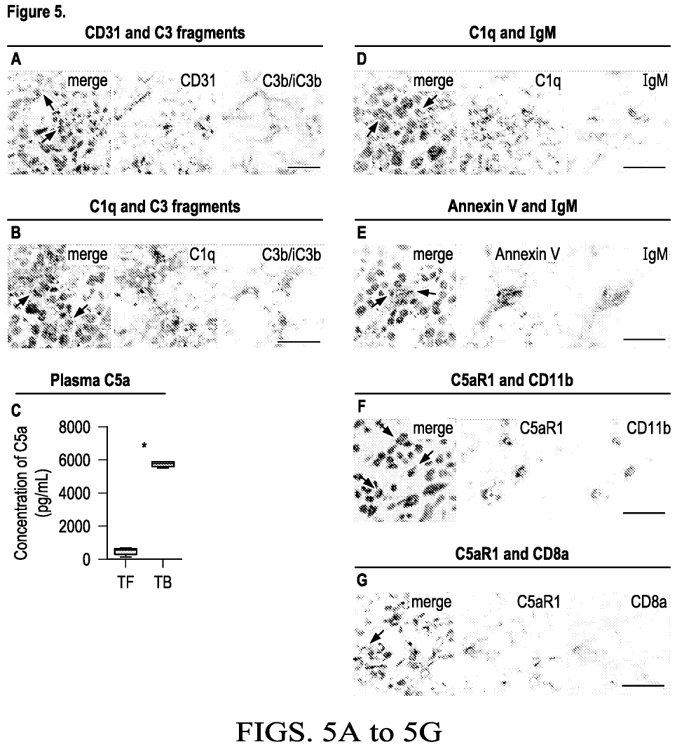

To test in vivo the role of complement proteins, the inventors used RCC-Renca (ATCC® CRL-2947™), a murine RCC model that while differing from human RCC, has been extensively evaluated in immunotherapy studies and is known to be recalcitrant to ICI [34], and therefore, potentially resembles ICI-resistant RCC. To evaluate the suitability of this model to study the role of complement, the inventors stained mouse tumors for complement proteins. C3 fragments were found in the vicinity of vasculature (FIG. 5A). C3 deposits colocalized with C1q (FIG. 5B), indicating a possible contribution of the classical pathway to the activation of complement [13]. C3 deposition without association with C1q likely indicates involvement of the alternative pathway because this pathway is initiated by spontaneous C3 hydrolysis followed by C3b deposition [35]. Mannose-binding lectin, which initiates the lectin complement pathway, was not found in these tumors (data not shown). Of note; the alternative pathway amplification loop was demonstrated to contribute to 80-90% of C5a generation, when complement is initiated through the classical pathway [36], and the inventors found higher amounts of C5a in plasma of tumor-bearing mice (TB) vs. tumor-free mice (TF, FIG. 5C). Because the classical pathway is initiated by C1q binding to immune complexes containing either IgG or IgM, with the latter having much greater capacity to activate complement, the inventors examined deposition of IgG and IgM along with C1q in mouse tumors. C1q colocalized with IgM (FIG. 5D) but not with IgG (data not shown), suggesting that IgM initiates complement activation. IgM deposits colocalized with Annexin V bound to apoptotic cells in mouse tumors (FIG. 5E). The inventors found numerous CD11b⁺ (myeloid) cells and CD8⁺ T cells expressing C5aR1 (FIGS. 5F, 5G), which is consistent with the role of C5aR1 in regulating MDSC [4] and TIL [37].

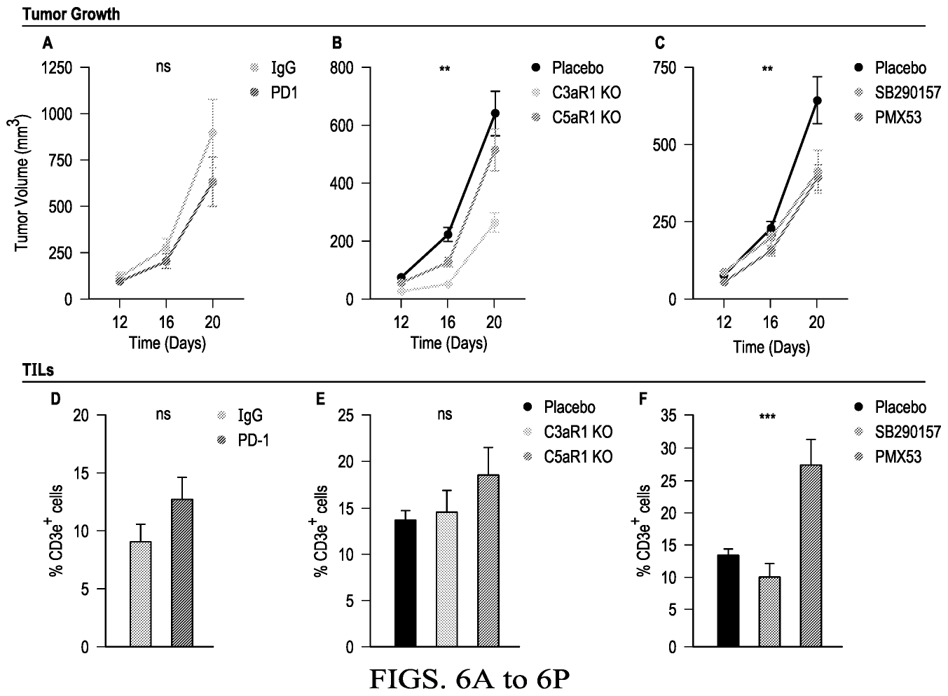

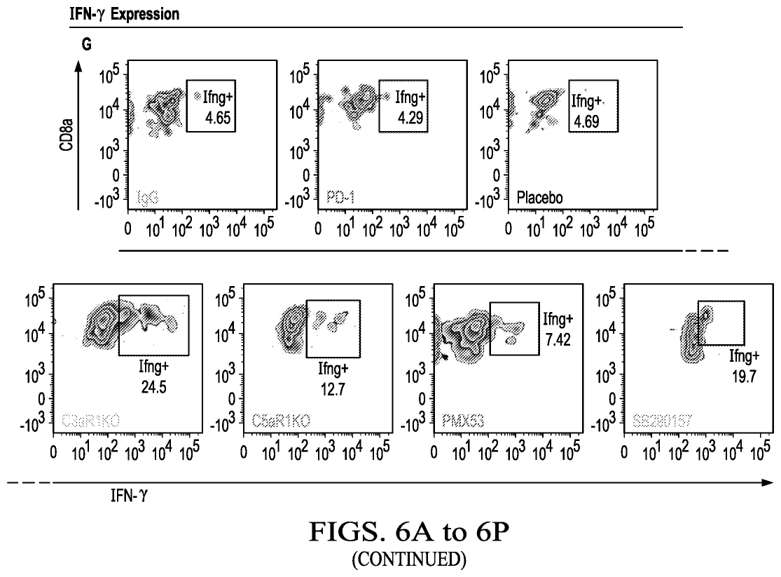

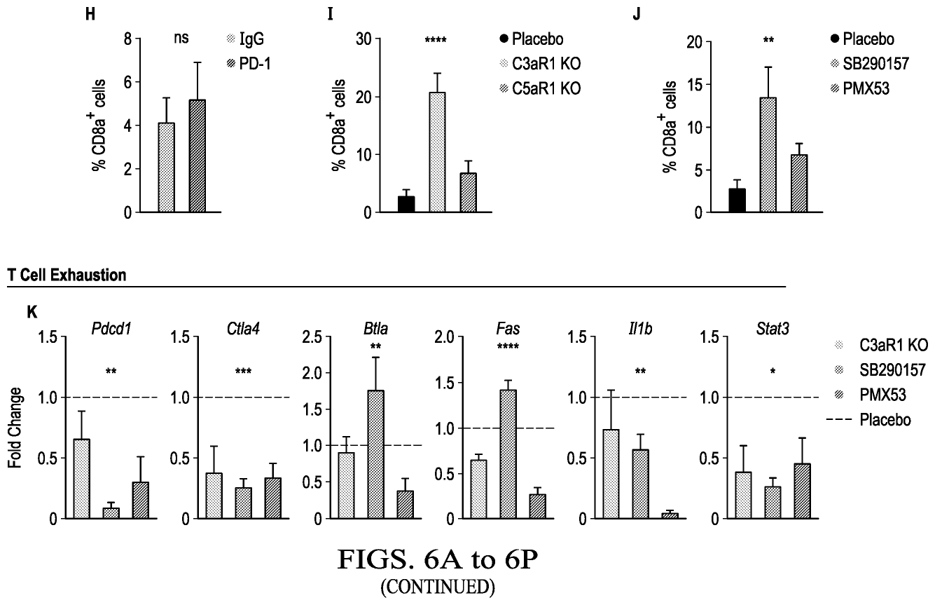

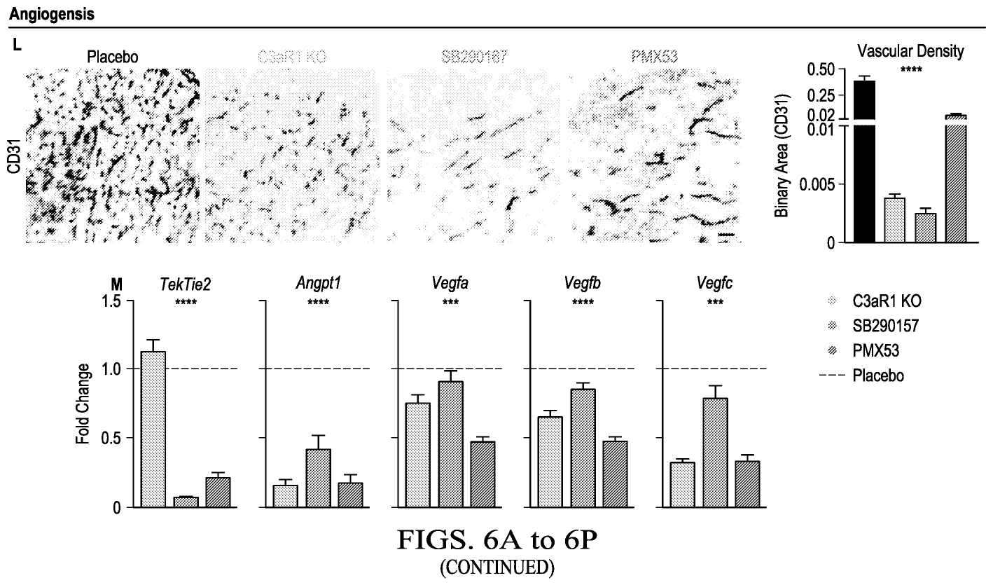

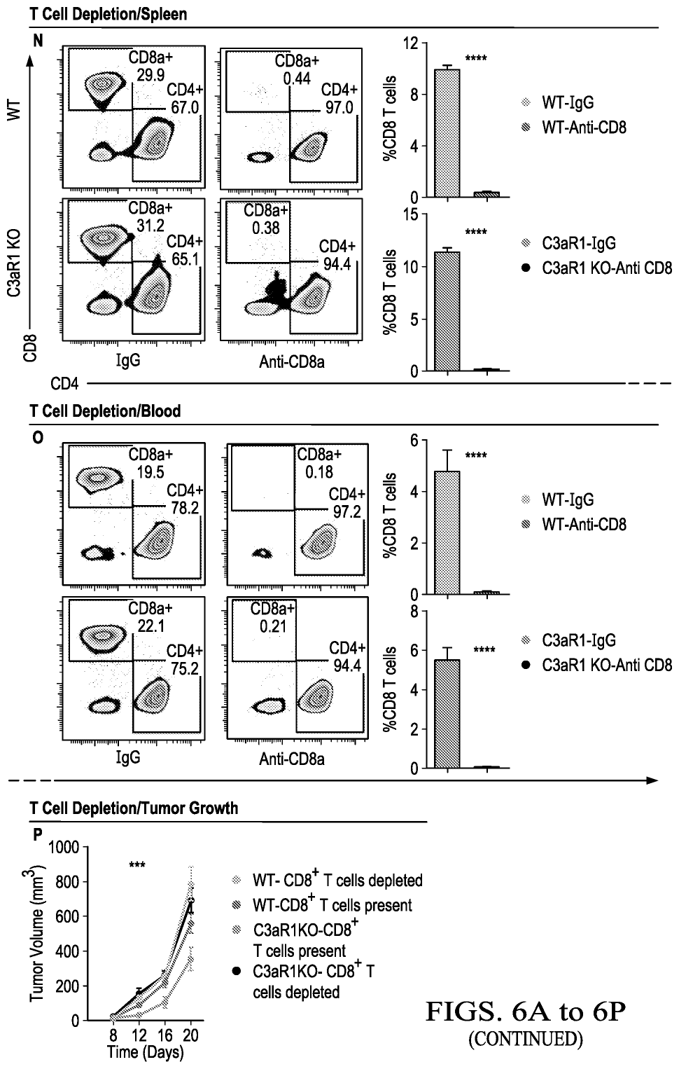

As previously reported [34], PD-1 blockade was ineffective in this model (FIG. 6A). In contrast, tumor growth was significantly impaired in C3aR1 knockouts (C3aR1 KO) (FIG. 6B). C5aR1 KO showed a trend for reduced tumor growth, although differences between wild-type mice injected with PBS (placebo) and C5aR1KO did not reach statistical significance. Consistent with the phenotypes in KO mice, wild-type mice treated with C3aR1 (SB290157) and C5aR1 (PMX53) inhibitors had reduced tumor growth (FIG. 6C). Because both C3aR1 and C5aR1 were implicated in suppressing antitumor T cell responses directly or indirectly [23, 37], the inventors investigated the impact of complement deficiencies/blockade on T-cells in tumors. As for anti-PD-1, C3aR1 or C5aR1-genetic deficiencies did not impact recruitment of CD8⁺ T cells to tumors (FIGS. 6D, 6E). However, pharmacological inhibition of C5aR1 resulted in influx CD8⁺ T cells to tumors (FIG. 6F), consistent with previous studies [4]. Unlike anti-PD-1 (FIGS. 6G, 6H), the genetic-deficiencies and pharmacological blockade of complement receptors improved TIL function, as demonstrated by increased production of IFN-γ upon ex-vivo restimulation (FIGS. 6G, 6I, and 6J). The highest production of IFN-γ was associated with lack of or blockade of C3aR1, corresponding to highest reduction of tumor growth in these mice. The increase in IFN-γ production suggests improved functionally and could indicate the reversal of T cell exhaustion, which, in the absence of treatment, is driven by immunosuppressive TME [27]. Therefore, the inventors next evaluated expression of genes associated with T cell exhaustion in mouse cohorts that exhibited significant phenotypes (C3aR1KO, SB290157, and PMX53). The inventors found down-regulation of T cell inhibitory pathways, indicated by a reduction in the expression of genes for T cell inhibitory receptors (Pdcd1, Ctla4, and Btla), cellular receptor Fas, and transcription factor Stat3 in C3aR1KO and PMX53 treated mice (FIG. 6K). Btla and Fas were upregulated in SB 290157-treated mice, reflecting multifaceted impact of these genes on T cell function. Although BTLA and Fas are well-recognized markers of T cell exhaustion [27], BTLA may also provide costimulatory signals to CD8⁺ T cells [38] and Fas/FasL signaling is critical for the survival of exhausted CD8⁺ T cells during immune response to tumors [39]. In accordance with recent work from the inventors demonstrating contributions of complement to angiogenesis [40], the inventors found that complement deficiency/blockade was associated with reduced vascular density in tumors (FIG. 6L) and reduced expression of proangiogenic factors (FIG. 6M), except Tie2 in a C3aR1KO. To confirm that the therapeutic effect of complement deficiencies is T cell-dependent, the inventors chose to deplete CD8⁺ T cells in C3aR1KO, as these mice had most profoundly reduced tumor growth (FIG. 6B). The treatment of mice with neutralizing anti-CD8α led to CD8⁺ T cell depletion, as demonstrated by lack of these cells in the spleen and peripheral blood of wild-type (WT) and C3aR1KO mice at the end point of this experiment (FIGS. 6N, 6O). As expected, injections of isotype-matched control IgG did not affect CD8⁺ T cell subsets (FIGS. 6N, 6O). The inventors challenged these mice and controls with Renca cells three days after the first dose of antiCD8α and IgG. C3aR1KO receiving control IgG, with CD8⁺ T cells present, had significantly reduced tumor growth when compared to IgG-treated WT controls, as expected (FIG. 6P). When CD8⁺ T cells were depleted from C3aR1KO, the beneficial impact of C3aR1-deficiency on tumor growth disappeared, as tumors grew in these mice at the same rate as in both WT cohorts (with or without CD8⁺ T cells) (FIG. 6P). Of note, the finding that tumor growth in WT mice with or without CD8⁺ T cells is similar suggests that C3aR1 renders T cells dysfunctional. Overall, these data show that CD8⁺ T cells are required for reduced tumor growth in C3aR1KO, suggesting contributions of this receptor to T cell-dysfunction in this RCC model.

For several decades, complement was thought to contribute to cancer immune surveillance through complement-mediated lysis of tumor cells [1]. In contrast, the inventors found that complement promotes tumor growth through the inhibition of antitumor immunity, mediated via activation and recruitment of MDSC to tumors [4]. Follow-up studies documented a critical role of complement in immunosuppression in several mouse models and discovered other mechanisms involved in this process [6]. In addition to regulating TME, complement proteins and receptors directly impact tumor cells [24]. The most recent studies established a link between complement and cancer metastasis [8, 9] and demonstrated synergism between PD-1 blockade and complement inhibition [41]. Thus, there is substantial evidence from preclinical studies pointing to complement as a potential therapeutic target in cancer. Furthermore, studies using human samples indicate that early complement components (C1q, C2, and C4) are prognostic biomarkers in lung and kidney cancer [13, 42]. However, there is limited understanding of complement in human malignancies and in vivo RCC models.

The inventors found that high expression of eleven complement genes was associated with unfavorable prognosis in RCC. In contrast to recent studies [13], the inventors' analysis of large data sets from TCGA and the Human Protein Atlas failed to demonstrate a prognostic role for C4 in RCC, underscoring the need for this comprehensive analysis. No other solid tumor evaluated had such striking correlation between complement gene expression and prognosis. Supporting the same notion, the inventors found that the MAC/TCC inhibitor CD59, which inhibits complement, was associated with good prognosis. These data also indicate that limiting the final stage of complement activation may be beneficial for RCC patients. In contrast to CD59, expression of another complement regulatory protein, CD55, was not associated with improved prognosis (https://www.proteinatlas.org/ENSG00000196352-CD55/pathology). In contrast to RCC, high expression of complement genes was associated with favorable prognosis in liver, breast, pancreatic, and cervical carcinomas. Therefore, inhibiting complement may not be universally beneficial for all cancer patients and RCC seems to be an optimal target for complement-based therapy. Immunohistochemistry data confirmed the presence of complement proteins in RCC tumors. However, it is unclear where else these proteins are produced. For example, they may be synthesized in the liver and deposited in the tumor stroma and vasculature.

Data from the UTSW KCP found an association between complement gene expression and an aggressive IS of RCC, which is consistent with key roles of several complement fragments in inflammation [22]. The correlation between complement and markers of T cell exhaustion/dysfunction and alternatively activated macrophages implicates complement in regulating immunosuppression in human RCC. This is supported by the fact that the inventors found reduced expression of genes associated with T cell exhaustion as a result of complement-deficiency/inhibition in a mouse model. The contribution of C3aR1 to T cell dysfunction is further corroborated by studies of T cell-depletion showing that intact CD8⁺ T cells are required to reduce tumor growth in C3aR1KO mice (i.e. C3aR1 loss improves T cell function). Based on these data, complement appears to act as an additional checkpoint in RCC, which corresponds to studies indicating roles of C3aR1 and C5aR1 in the regulation of cytolytic activity of TIL in other mouse models [37]. Therefore, it is conceivable that in the presence of complement-imposed immunosuppression, therapeutic inhibition of the PD-1/CTLA-4 pathways will be ineffective. The resistance to ICI in patients with high TCC levels and low C5 levels suggest that complement activation, and possibly, subsequent complement consumption, plays a role in RCC pathogenesis. This consumption is best characterized in autoimmune diseases such as systemic lupus erythematosus and urticarial vasculitis, in which hypocomplementemia supports the diagnosis and is used to monitor disease activity [43]. Because the C1q gene signature is strongly associated with poor prognosis and aggressive IS of RCC and C1q is deposited in human and mouse tumors, the contribution of the classical pathway of complement activation to RCC should be considered. C1q colocalized with IgM in mouse tumors, therefore, the inventors theorize that these IgM may trigger the classical pathway. These IgMs may represent poly-reactive natural antibodies constitutively present in high quantities in the body fluids that bind endogenous antigens in dying, damaged, or otherwise stressed cells [44, 45]. Of note, IgM decorated apoptotic cell marked by Annexin V in mouse RCC model.

In conclusion, complement is involved in RCC pathogenesis and impacts antitumor immunity, which is reflected by its association with inflammation and poor prognosis. Targeting complement is a therapeutic option for RCC patients.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB.

Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1. Markiewski, M. M. and J. D. Lambris, Is complement good or bad for cancer patients? A new perspective on an old dilemma. Trends Immunol, 2009. 30(6): p. 286-92.
2. Fishelson, Z., et al., Obstacles to cancer immunotherapy: expression of membrane complement regulatory proteins (mCRPs) in tumors. Mol Immunol, 2003. 40(2-4): p. 109-23.
3. Taylor. R. P. and M. A. Lindorfer, Cytotoxic mechanisms of immunotherapy: Harnessing complement in the action of anti-tumor monoclonal antibodies. Semin Immunol, 2016. 28(3): p. 309-16.
4. Markiewski, M. M., et al., Modulation of the antitumor immune response by complement. Nat Immunol, 2008. 9(11): p. 1225-35.
5. Markiewski, M. M. and J. D. Lambris, Unwelcome complement. Cancer Res, 2009. 69(16): p. 6367-70.
6. Reis, E. S., et al., Complement in cancer: untangling an intricate relationship. Nat Rev Immunol, 2017.
7. Afshar-Kharghan, V., The role of the complement system in cancer. J Clin Invest, 2017. 127(3): p. 780-789.
8. Vadrevu, S. K., et al., Complement c5a receptor facilitates cancer metastasis by altering T-cell responses in the metastatic niche. Cancer Res, 2014. 74(13): p. 3454-65.
9. Sharma, S. K., et al., Pulmonary alveolar macrophages contribute to the premetastatic niche by suppressing antitumor T cell responses in the lungs. J Immunol, 2015. 194(11): p. 5529-38.
10. Kochanek, D. M., et al., Complementing Cancer Metastasis. Front Immunol, 2018. 9: p. 1629.
11. Ajona, D., et al., A Combined PD-1/C5a Blockade Synergistically Protects Against Lung Cancer Growth and Metastasis. Cancer Discov, 2017.
12. Xi, W., et al., High Level of Anaphylatoxin C5a Predicts Poor Clinical Outcome in Patients with Clear Cell Renal Cell Carcinoma. Scientific Reports, 2016. 6.
13. Roumenina, L. T., et al., Tumor Cells Hijack Macrophage-Produced Complement C1q to Promote Tumor Growth. Cancer Immunol Res, 2019. 7(7): p. 1091-1105.
14. Ricklin, D., A. Barratt-Due, and T. E. Mollnes, Complement in clinical medicine: Clinical trials, case reports and therapy monitoring. Mol Immunol, 2017. 89: p. 10-21.
15. Wang, T., et al., An Empirical Approach Leveraging Tumorgrafts to Dissect the Tumor Microenvironment in Renal Cell Carcinoma Identifies Missing Link to Prognostic Inflammatory Factors. Cancer Discov, 2018. 8(9): p. 1142-1155.
16. Delahunt, B., et al., The International Society of Urological Pathology (ISUP) grading system for renal cell carcinoma and other prognostic parameters. Am J Surg Pathol, 2013. 37(10): p. 1490-504.
17. Monk, P. N., et al., Function, structure and therapeutic potential of complement C5a receptors. Br J Pharmacol, 2007. 152(4): p. 429-48.
18. Ngiow, S. F., et al., A Threshold Level of Intratumor CD8+ T-cell PD1 Expression Dictates Therapeutic Response to Anti-PD1. Cancer Res, 2015. 75(18): p. 3800-11.
19. Markowitz, G. J., et al., Immune reprogramming via PD-1 inhibition enhances early-stage lung cancer survival. JCI Insight, 2018. 3(13).
20. Mastellos, D., et al., Novel monoclonal antibodies against mouse C3 interfering with complement activation: description of fine specificity and applications to various immunoassays. Mol Immunol, 2004. 40(16): p. 1213-21.
21. Pedregosa, F., et al., Scikit-learn: Machine Learning in Python. Journal of Machine Learning Research, 2011. 12: p. 2825-2830.
22. Markiewski, M. M. and J. D. Lambris, The role of complement in inflammatory diseases from behind the scenes into the spotlight. Am J Pathol, 2007. 171(3): p. 715-27.
23. Kolev, M. and M. M. Markiewski, Targeting complement-mediated immunoregulation for cancer immunotherapy. Semin Immunol, 2018.
24. Cho, M. S., et al., Autocrine effects of tumor-derived complement. Cell Rep, 2014. 6(6): p. 1085-95.
25. Feldmeyer, L., O. Gaide, and D. E. Speiser, Clinical Implications of CD8+ T-Cell Infiltration in Frequent and Rare Cancers. Journal of Investigative Dermatology, 2013. 133(8): p. 1929-1932.
26. Chow, M. T. and A. D. Luster, Chemokines in cancer. Cancer Immunol Res, 2014. 2(12): p. 1125-31.
27. Blank, C. U., et al., Defining ‘T cell exhaustion’. Nat Rev Immunol, 2019. 19(11): p. 665-674.
28. Smith-Garvin, J. E., G. A. Koretzky, and M. S. Jordan, T cell activation. Annu Rev Immunol, 2009. 27: p. 591-619.
29. Ugel, S., et al., Tumor-induced myeloid deviation: when myeloid-derived suppressor cells meet tumor-associated macrophages. J Clin Invest, 2015. 125(9): p. 3365-76.
30. Murray, P. J., et al., Macrophage activation and polarization: nomenclature and experimental guidelines. Immunity, 2014. 41(1): p. 14-20.
31. Teng, M. W., et al., Classifying Cancers Based on T-cell Infiltration and PD-L1. Cancer Res, 2015. 75(11): p. 2139-45.
32. Prohaszka, Z., et al., Complement analysis 2016: Clinical indications, laboratory diagnostics and quality control. Immunobiology, 2016. 221(11): p. 1247-58.
33. Jaworski, M., et al., New Splitting Criteria for Decision Trees in Stationary Data Streams. IEEE Trans Neural Netw Learn Syst, 2018. 29(6): p. 2516-2529.
34. O'Shaughnessy, M. J., et al., Systemic Antitumor Immunity by PD-1/PD-L1 Inhibition Is Potentiated by Vascular-Targeted Photodynamic Therapy of Primary Tumors. Clin Cancer Res, 2018. 24(3): p. 592-599.
35. Thurman, J. M. and V. M. Holers, The central role of the alternative complement pathway in human disease. J Immunol, 2006. 176(3): p. 1305-10.
36. Harboe, M. and T. E. Mollnes, The alternative complement pathway revisited. J Cell Mol Med, 2008. 12(4): p. 1074-84.
37. Wang, Y., et al., Autocrine Complement Inhibits IL10-Dependent T-cell-Mediated Antitumor Immunity to Promote Tumor Progression. Cancer Discov, 2016. 6(9): p. 1022-35.
38. Ritthipichai, K., et al., Multifaceted Role of BTLA in the Control of CD8(+) T-cell Fate after Antigen Encounter. Clin Cancer Res, 2017. 23(20): p. 6151-6164.
39. Yajima, T., et al., Fas/FasL signaling is critical for the survival of exhausted antigen-specific CD8(+) T cells during tumor immune response. Mol Immunol, 2019. 107: p. 97-105.
40. Ghouse, S. M., et al., Therapeutic Targeting of Vasculature in the Premetastatic and Metastatic Niches Reduces Lung Metastasis. J Immunol, 2020.
41. Ajona, D., et al., A Combined PD-1/C5a Blockade Synergistically Protects against Lung Cancer Growth and Metastasis. Cancer Discov, 2017. 7(7): p. 694-703.
42. Ajona, D., et al., Investigation of complement activation product c4d as a diagnostic and prognostic biomarker for lung cancer. J Natl Cancer Inst, 2013. 105(18): p. 1385-93.
43. Nilsson, B. and K. N. Ekdahl, Complement diagnostics: concepts, indications, and practical guidelines. Clin Dev Immunol, 2012. 2012: p. 962702.
44. Ehrenstein, M. R. and C. A. Notley, The importance of natural IgM: scavenger, protector and regulator. Nat Rev Immunol, 2010. 10(11): p. 778-86.
45. Wang, H., J. E. Coligan, and H. C. Morse, 3rd, Emerging Functions of Natural IgM and Its Fc Receptor FCMR in Immune Homeostasis. Front Immunol, 2016. 7: p. 99.

1. A method of determining a prognosis of a subject with cancer comprising:
obtaining or having obtained a sample from the subject; and
measuring in the sample a level of expression of one or more Complement or Complement related genes or proteins; and
determining if the levels of expression of the Complement or Complement related gene or protein when compared to the levels of expression of the Complement or Complement related genes or proteins from a subject that does not have cancer, wherein a change in the level of expression of the Complement or Complement related genes or proteins is associated with an unfavorable prognosis or a favorable prognosis.

2. The method of claim 1, wherein the cancer is selected from renal, urothelial, stomach, liver, pancreatic, breast, head/neck, testis, ovarian, and cervical.

3. The method of claim 1, wherein the Complement or Complement related gene or protein is selected from C1QA, C1QB, C1S, C1R, C2, C3, C5, C6, C7, C8B, CFB, CFD, CFH, CFI, CD21/CR2, CD46, CD55, CD59, C5AR1.

4. The method of claim 1, wherein the Complement or Complement related gene or protein is favorable and is selected from at least one of:

| Complement Gene | Cancer Type | Prognosis |
|---|---|---|
| C1S | Liver | Favorable |
| C3 | Liver | Favorable |
| C5 | Liver | Favorable |
| C6 | Liver | Favorable |
| C7 | liver | Favorable |
| C8B | Liver | Favorable |
| CFB | Breast | Favorable |
| CFD | Pancreatic | Favorable |
| CD21/CR2 | Breast | Favorable |
| CD46 | Stomach | Favorable |
| CD59 | Renal | Favorable |
| C5AR1 | Cervical | Favorable[[.]]; |

or
wherein the Complement or Complement related gene or protein is unfavorable and is selected from at least one of:

| Complement Gene | Cancer Type | Prognosis |
|---|---|---|
| C1QA | Renal | Unfavorable |
| C1QB | Renal | Unfavorable |
| C1S | Renal | Unfavorable |
| C1R | Renal | Unfavorable |
| C2 | Renal | Unfavorable |
| C3 | Renal | Unfavorable |
| CFB | Renal | Unfavorable |
| CFD | Renal | Unfavorable |
| CFH | Renal | Unfavorable |
| CFI | Urothelial | Unfavorable |
| CD46 | Cervical | Unfavorable |
| CD55 | Renal | Unfavorable |
| CD59 | Pancreatic | Unfavorable |
| | Head/Neck | Unfavorable |
| | Cervical | Unfavorable |
| C5AR1 | Renal | Unfavorable |
| | Testis | Unfavorable |
| | Ovarian | Unfavorable. |

5. (canceled)

6. The method of claim 1, wherein a histological grade of the cancer is determined by the expressed or deposition of Complement proteins in tumor stroma.

7. The method of claim 1, wherein the Complement or Complement related gene or protein CFB, C5AR1, CFH, C3, C1R, C1S C1QA, and C1QB are enriched in aggressive inflammatory phenotype cancers.

8. The method of claim 1, further comprising determining a level of expression of macrophage biomarkers selected from CD86, IRF1, STAB1, TFGB1, F13A1, IL-6, and CD40, wherein expression of one or more of the macrophage biomarkers is associated with an unfavorable prognosis.

9. The method of claim 1, wherein the sample is a plasma sample.

10. The method of claim 1, further comprising separating a subject into a those with a higher or a lower level of expression of the Complement or Complement related gene or protein, and:
if the subject has low FH and FD expression the subject has a worse response to an immune checkpoint inhibitor;
if the subject has low FI and TCC the subject has a better response to an immune checkpoint inhibitor; or
if the subject has low TCC and high C5 the subject has a better response to an immune checkpoint inhibitor.

11. The method of claim 10, wherein the immune checkpoint inhibitor is selected from nivolumab, ipilimumab, tremelimumab, ipilimumab and nivolumab, pembrolizumab, nivolumab, pidilizumab, MK-3475, MED 14736, CT-011, spartalizumab, durvalumab, atezolizumab, avelumab, AMP224, BMS-936559, MPLDL3280A, or MSB0010718C, or is selected from inhibitors of at least one of: CD137, CD134, PD-1, KIR, LAG-3, PD-L1, PDL2, CTLA-4, B7.1, B7.2, B7-DC, B7-H1, B7-H2, B7-H3, B7-H4, B7-H5, B7-H6, B7-H7, BTLA, LIGHT, HVEM, GALS, TIM-3, TIGHT, VISTA, 2B4, CGEN-15049, CHK 1, CHK2, A2aR, TGF-beta, PI3Kgamma, GITR, ICOS, IDO, TLR, IL-2R, IL-10, PVRIG, CCRY, OX-40, CD160, CD20, CD52, CD47, CD73, CD27-CD70, or CD40.

12. (canceled)

13. The method of claim 1, further comprising the step of treating a renal cell carcinoma with treated with C3aR1 and C5aR1 inhibitors to reduce tumor growth; or
treating the subject with a complement blockade to at least one of: reduce vascular density in tumors or reduce expression of proangiogenic factors.

14. (canceled)

15. A method of treating a subject with cancer comprising:
obtaining or having obtained a sample from the subject; and
measuring in the sample a level of expression of one or more Complement or Complement related genes or proteins;
determining if the levels of expression of the Complement or Complement related gene or protein when compared to the levels of expression of the Complement or Complement related genes or proteins from a subject that does not have cancer, wherein a change in the level of expression of the Complement or Complement related genes or proteins is associated with an unfavorable prognosis or a favorable prognosis; and
if the subject has low FH and FD expression the subject has a worse response to an immune checkpoint inhibitor;
if the subject has low FI and TCC the subject has a better response to an immune checkpoint inhibitor; or
if the subject has low TCC and high C5 the subject has a better response to an immune checkpoint inhibitor, wherein the cancer is selected from renal, urothelial, stomach, liver, pancreatic, breast, head/neck, testis, ovarian, and cervical.

16. The method of claim 15, wherein the immune checkpoint inhibitor is selected from nivolumab, ipilimumab, tremelimumab, ipilimumab and nivolumab, pembrolizumab, nivolumab, pidilizumab, MK-3475, MED 14736, CT-011, spartalizumab, durvalumab, atezolizumab, avelumab, AMP224, BMS-936559, MPLDL3280A, or MSB0010718C, or is selected from inhibitors of at least one of: CD137, CD134, PD-1, KIR, LAG-3, PD-L1, PDL2, CTLA-4, B7.1, B7.2, B7-DC, B7-H1, B7-H2, B7-H3, B7-H4, B7-H5, B7-H6, B7-H7, BTLA, LIGHT, HVEM, GALS, TIM-3, TIGHT, VISTA, 2B4, CGEN-15049, CHK 1, CHK2, A2aR, TGF-beta, PI3Kgamma, GITR, ICOS, IDO, TLR, IL-2R, IL-10, PVRIG, CCRY, OX-40, CD160, CD20, CD52, CD47, CD73, CD27-CD70, or CD40.

17. (canceled)

18. (canceled)

19. The method of claim 15, wherein a histological grade of the cancer is determined by the expressed or deposition of Complement proteins in tumor stroma.

20. The method of claim 15, wherein the Complement or Complement related gene or protein CFB, C5AR1, CFH, C3, CIR, CIS C1QA, and C1QB are enriched in aggressive inflammatory phenotype cancers.

21. The method of claim 15, further comprising determining a level of expression of macrophage biomarkers selected from CD86, IRF1, STAB1, TFGB1, F13A1, IL-6, and CD40, wherein expression of one or more of the macrophage biomarkers is associated with an unfavorable prognosis.

22. The method of claim 15, wherein the sample is a plasma sample.

23. The method of claim 15, further comprising the step of treating a renal cell carcinoma with treated with C3aR1 and C5aR1 inhibitors to reduce tumor growth; or
treating the subject with a complement blockade to at least one of: reduce vascular density in tumors or reduce expression of proangiogenic factors.

24. (canceled)

25. A method for treating a cancer comprising the steps of:
performing or having performed a level of expression of one or more Complement or Complement related genes or proteins;
determining if the levels of expression of the Complement or Complement related gene or protein when compared to the levels of expression of the Complement or Complement related genes or proteins from a subject that does not have cancer, wherein a change in the level of expression of the Complement or Complement related genes or proteins is associated with an unfavorable prognosis or a favorable prognosis; and
if the subject has low FH and FD expression the subject has a worse response to an immune checkpoint inhibitor;
if the subject has low FI and TCC the subject has a better response to an immune checkpoint inhibitor; or
if the subject has low TCC and high C5 the subject has a better response to an immune checkpoint inhibitor.

26. The method of claim 25, wherein the immune checkpoint inhibitor is selected from nivolumab, ipilimumab, tremelimumab, ipilimumab and nivolumab, pembrolizumab, nivolumab, pidilizumab, MK-3475, MED 14736, CT-011, spartalizumab, durvalumab, atezolizumab, avelumab, AMP224, BMS-936559, MPLDL3280A, or MSB0010718C; or wherein the immune checkpoint inhibitor is selected from inhibitors of at least one of: CD137, CD134, PD-1, KIR, LAG-3, PD-L1, PDL2, CTLA-4, B7.1, B7.2, B7-DC, B7-H1, B7-H2, B7-H3, B7-H4, B7-H5, B7-H6, B7-H7, BTLA, LIGHT, HVEM, GALS, TIM-3, TIGHT, VISTA, 2B4, CGEN-15049, CHK 1, CHK2, A2aR, TGF-beta, PI3Kgamma, GITR, ICOS, IDO, TLR, IL-2R, IL-10, PVRIG, CCRY, OX-40, CD160, CD20, CD52, CD47, CD73, CD27-CD70, or CD40.

27. (canceled)
